(19) United States
(12) Patent Application Publication
(10) Pub. No.: US 2011/0028929 A1
(43) Pub. Date: Feb. 3, 2011

(54) Absorbent Products with Beneficial Indicia

(76) Inventors: Heidi Bauerlein Hopkins, Neenah, WI (US); Adrienne Rae Loyd, Neenah, WI (US); Suzanne K. Sullivan, Appleton, WI (US); Katie Ann Boland, Neenah, WI (US)

Correspondence Address: KIMBERLY-CLARK WORLDWIDE, INC.;Tara Pohlkotte, 2300 Winchester Rd., NEENAH, WI, 54956, US

(21) Appl. No.: 12/645,975

(22) Filed: Dec. 23, 2009

Related U.S. Application Data
(60) Provisional application No. 61/230,915, filed on Aug. 3, 2009.

(51) Int. Cl. A61F 13/45; A61F 13/15

(52) U.S. Cl. 604/383; 604/385.02; 604/385.03

(57) ABSTRACT

A system for visually identifying the performance level of feminine care pads comprises a first feminine care pad and a second feminine care pad, each having a body-facing surface, a garment-facing surface, and a printed pattern that is visible to a user on at least one of the body-facing surface or the garment-facing surface. The first feminine care pad and the second feminine care pad are related. The second feminine care pad has a higher absorbent capacity level than the first feminine care pad. In addition, the total printed surface area of the printed pattern of the second feminine care pad is greater than the total printed surface area of the printed pattern of the first feminine care pad.

### PRIORITY

This non-provisional application claims priority of Provisional Application No. 61/230,915, filed on Aug. 3, 2009. The entirety of Application No. 61/230,915 is incorporated herein by reference.

### BACKGROUND

Personal care articles are well known in the art. Such articles can desirably be disposable. Such articles can also be absorbent and can absorb discharged bodily fluids from a user. Absorbent articles generally comprise a fibrous mass or other absorbent structure which can absorb and hold body fluids. Similarly, it is well known that feminine care articles have been employed to absorb and hold liquids, such as urine and complex fluids such as menses. A typical structure of an absorbent article can optionally include a fluid impermeable backsheet (also called a baffle or garment-side liner), a fluid permeable topsheet (also called a cover or body-side liner) and an absorbent core (also called an absorbent structure) positioned between the backsheet and the topsheet. Prior absorbent articles have also included various other features to improve fluid handling, such as intake layers, distribution layers, retention layers and the like. In these disposable personal care absorbent articles, the topsheet is the body-facing side of the absorbent article and the backsheet is the garment-facing side of the absorbent article.

In the case of feminine pads, for example, wearers tend to experience different volumes of menses excretion during the menstrual cycle. As a result, a variety of feminine pads having different performance values, such as absorbent capacity for example, are available to meet the needs of the user. Such feminine articles can range from pantiliners having a relatively low performance value for light menstrual days, to ultrathin pads having a higher performance value for moderate menstrual days, to maxi-pads having an even higher performance value for heavy flow days.

As technology has progressed over time, disposable personal care absorbent articles have become substantially thinner and often more compact. As a result, the variety of related articles (such as feminine care pads, for example) have become more visually similar, making it difficult to distinguish between articles having different performance values. Attempts have been made to alleviate this problem by placing indicia on the packaging for absorbent articles which distinguish performance level. However, this does not alleviate the problem when such articles are present without packaging. The problem is exacerbated when two or more articles having differing performance values are present.

Thus, there is a need for a system for visually identifying the performance level of disposable personal care absorbent articles, particularly when two or more such articles are present having differing performance values.

Additionally, it can be desirable to invoke an emotional response from the user of an absorbent article due to the design and/or marketing of the article. Further discussion of invoking an emotion response through article design and/or marketing can be found in the article “Predefining Emotions for Visual Product Appearance” by Nicolas et al., found at http://www.mooldesign.com/articles/Predefining_emotions_for_visual_product_appearance.pdf. For example, the past and current marketing of feminine-hygiene products has resulted in a culture that associates menstruation with a medical or clinical condition, and has made users often feel unclean, self-conscious and even embarrassed. Even the phrases “feminine-hygiene product” and “vaginal-health product,” and words that describe these products and their use, such as “cycle” or “protection,” are suggestive of this culture. As a result, women view menstruation and vaginal health as medical in nature, perhaps something that is not normal, rather than a natural part of who they are. This association between feminine-hygiene products and the current culture so shapes a user's perception of these products that she may be reluctant to talk openly about her body, menstruation, and vaginal health. This is unfortunate because the more a woman understands herself, and what is going on with her body, the better.

Also, this historic association between feminine-hygiene products and the current culture may conflict with how some women view themselves and their bodies. However, if a woman could move beyond the taboos associated with vaginal health and, instead, perceive herself as open and in command and that vaginal health, including menstruation, is a part of who she is, then the current culture could be viewed as outdated and irrelevant. In addition, if a manufacturer's choice of words, images, and color expressly or implicitly links these products to what is perceived as the current culture, then these same women may reject the manufacturer's products.

As a result of the current culture, manufacturers have primarily focused on feminine care articles that invoke an emotional response of being or feeling “discrete”. However, the focus on discreteness has unfortunately further reinforced the current culture of sensitivity, privacy and embarrassment felt by women. Some manufacturers have used product names that, by themselves, do not necessarily suggest a medical or clinical condition. For example, a number of companies have used product names that suggest the word “free”—possibly to suggest, at least some decades ago, freedom from supporting belts that were once used to hold pads in place: STAYFREE-brand feminine-care products; CAREFREE-brand feminine-care products; FREEDOM-brand feminine-care products; NEW FREEDOM-brand feminine-care products; and others. Other manufacturers have used product names that suggest jewelry (“PEARL-brand feminine-care products”); an ongoing presence or availability (“ALWAYS-brand feminine-care products” and “WHENEVER-brand feminine-care products”); comfort (“GENTLE GLIDE-brand feminine-care products”); an unquantifiable number (“INFINITY-brand feminine-care products”); and athletics (“SPORT-brand feminine-care products”). None of these names, however, alone or as part of a larger product line, correspond to the bold, non-traditional product-name indicia needed to more completely break with the current culture of sensitivity, privacy and embarrassment.

Therefore, there is a desperate need for such absorbent articles which break the paradigms associated with the current culture as it relates to menstruation.

### SUMMARY

In response to the needs discussed above, a system for visually identifying the performance level of disposable personal care absorbent articles is provided. In some aspects, a system for visually identifying the performance level of feminine care pads comprises a first feminine care pad and a second feminine care pad, each having a body-facing surface, a garment-facing surface, and a printed pattern that is visible to a user on at least one of the body-facing surface or the garment-facing surface. The first feminine care pad and the second feminine care pad are related. The second feminine care pad has a higher absorbent capacity level than the first feminine care pad, as measured by the Absorbent Capacity Test. In addition, the total printed surface area of the printed pattern of the second feminine care pad is greater than the total printed surface area of the printed pattern of the first feminine care pad. In further aspects, the total surface area of the printed pattern of the second feminine care pad is at least 5% greater than the total surface area of the printed pattern of the first feminine care pad. In still further aspects, the total surface area the printed pattern of the second feminine care pad is 30%-800% greater than the total surface area of the printed pattern of the first feminine care pad. In yet further aspects, the printed pattern of the first feminine care pad and the printed pattern of the second feminine care pad are the same color. In still further aspects, the printed pattern of the first feminine care pad and the printed pattern of the second feminine care pad each comprise a color that is bold, vivid or brilliant. In yet further aspects, the printed pattern of the first feminine care pad and the printed pattern of the second feminine care pad are visually coordinated. In still further aspects, the first feminine care pad and the second feminine care pad further comprise embossing, wherein the total surface area of the printed pattern and embossing of the second feminine care pad is greater than the total surface area of the printed pattern and embossing of the first feminine care pad. In yet further aspects, the printed pattern of the first feminine care pad and the printed pattern of the second feminine care pad further comprise an optical illusion. In still further aspects, the printed pattern of the first feminine care pad and the printed pattern of the second feminine care pad each comprise multiple shades of the same color. In yet further aspects, the first feminine care pad and the second feminine care pad each comprise a wrapper, wherein each respective wrapper includes a printed pattern that is substantially the same as the printed pattern of the corresponding feminine care pad. In still further aspects, the system further comprises a first package and a second package, wherein the first package contains a plurality of the first feminine care pad and includes a printed pattern that is visually coordinated with the printed pattern of the first feminine care pad, and wherein the second package contains a plurality of the second feminine care pad and includes a printed pattern that is visually coordinated with the printed pattern of the second feminine care pad. In yet further aspects, the system further comprises a communication means including at least one scale of reference. In still further aspects, the absorbent capacity is saturated capacity. In yet further aspects, the absorbent capacity is retention capacity.

In some aspects, a system for visually identifying the performance level of feminine care pads comprises a first feminine care pad and a second feminine care pad, each having a body-facing surface, a garment-facing surface and a pattern that is visible to a user on the body-facing surface. The first feminine care pad and the second feminine care pad are related. In addition, each pattern comprises printing, wherein the printing of the first feminine care pad and the printing of the second feminine care pad are the same color, wherein the color is bold, brilliant or vivid. Furthermore, the saturation capacity of the second feminine care pad is greater than the saturation capacity of the first feminine care pad, as measured by the Absorbent Capacity Test. In addition, the total surface area of the pattern of the second feminine care pad is at least 10% greater than the total surface area of the pattern of the first feminine care pad. In further aspects, the total surface area of the pattern of the second feminine care pad is at least 50% greater than the total surface area of the printing of the pattern of the first feminine care pad. In still further aspects, the first feminine care pad and the second feminine care pad each further comprise a wrapper, wherein each respective wrapper includes a printed pattern that is substantially the same as the pattern of the corresponding feminine care pad. In yet further aspects, the system further comprises a first package and a second package, wherein the first package contains a plurality of the first feminine care pad and includes a printed pattern that is visually coordinated with the pattern of the first feminine care pad, and wherein the second package contains a plurality of the second feminine care pad and includes a printed pattern that is visually coordinated with the pattern of the second feminine care pad. In still further aspects, the system further comprises a communication means including at least one scale of reference.

In some aspects, a method for visually identifying the performance level of disposable personal care absorbent articles comprises a) providing a first disposable personal care absorbent article having a body-facing surface and a garment-facing surface; b) providing a second disposable personal care absorbent article having a body-facing surface and a garment-facing surface; and c) disposing a printed pattern that is visible to a user on at least one of the body-facing surface or the garment-facing surface on each of the first disposable personal care absorbent article and the second disposable personal care absorbent article. The first disposable personal care absorbent article and the second disposable personal care absorbent article are related.

In addition, the second disposable personal care absorbent article has a performance value that is greater than the first disposable personal care absorbent article. Furthermore, the total surface area of printed pattern of the second disposable personal care absorbent article is greater than the total surface area of the printed pattern of the first disposable personal care absorbent article. In further aspects, the printed pattern of the first disposable personal care absorbent article and the printed pattern of the second disposable personal care absorbent article are visually coordinated. In still further aspects, the printed pattern of the first disposable personal care absorbent article and the printed pattern of the second disposable personal care absorbent article each comprise a color that is bold, vivid or brilliant. In yet further aspects, the performance value is saturation capacity, as measured by the Absorbent Capacity Test. In still further aspects, the personal care absorbent articles are feminine care pads.

Numerous other features and advantages of the present invention will appear from the following description. In the description, reference is made to exemplary embodiments of the invention. Such embodiments do not represent the full scope of the invention. Reference should therefore be made to the claims herein for interpreting the full scope of the invention. In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

### FIGURES

The foregoing and other features, aspects and advantages of the present invention will become better understood with regard to the following description, appended claims and accompanying drawings where:

FIG. 1 is a top plan of a feminine napkin of the present disclosure.

FIG. 2 is a bottom plan of the napkin.

FIG. 3 is a section taken in the plane of line 3-3 of FIG. 1.

FIG. 4 is a top plan of another feminine napkin of the present disclosure having wings.

FIG. 5 is a bottom plan of the napkin shown in FIG. 4.

FIG. 6 is a section taken in the plane of line 6-6 of FIG. 4.

FIG. 7 is one version of a diagram used to characterize color which shows the Hunter L,a,b color space.

FIG. 8A is a top view of an invisible feel liner feminine pad having a performance identification device in the form of a fun print pattern.

FIG. 8B is a top view of a body contour liner feminine pad having a performance identification device in the form of a fun print pattern.

FIG. 8C is a top view of an ultrathin feminine pad having a performance identification device in the form of a fun print pattern.

FIG. 8D is a top view of a heavy flow feminine pad having a performance identification device in the form of a fun print pattern.

FIG. 8E is a top view of an overnight feminine pad having a performance identification device in the form of a fun print pattern.

FIG. 9A is a top view of an invisible feel liner feminine pad having a performance identification device in the form of a fun print pattern.

FIG. 9B is a top view of a body contour liner feminine pad having a performance identification device in the form of a fun print pattern.

FIG. 9C is a top view of an ultrathin feminine pad having a performance identification device in the form of a fun print pattern.

FIG. 9D is a top view of a heavy flow feminine pad having a performance identification device in the form of a fun print pattern.

FIG. 9E is a top view of an overnight feminine pad having a performance identification device in the form of a fun print pattern.

FIG. 10A is a top view of an invisible feel liner feminine pad having a performance identification device in the form of a fun print pattern.

FIG. 10B is a top view of a body contour liner feminine pad having a performance identification device in the form of a fun print pattern.

FIG. 10C is a top view of an ultrathin feminine pad having a performance identification device in the form of a fun print pattern.

FIG. 10D is a top view of a heavy flow feminine pad having a performance identification device in the form of a fun print pattern.

FIG. 10E is a top view of an overnight feminine pad having a performance identification device in the form of a fun print pattern.

FIG. 11 is an exemplary optical illusion suitable for use with the invention.

FIG. 12 is an exemplary optical illusion suitable for use with the invention.

FIG. 13 is an exemplary optical illusion suitable for use with the invention.

FIG. 14 is an exemplary folded article and wrapper configuration.

FIG. 15 is an exemplary folded article and wrapper configuration.

FIG. 16 is an exemplary folded article and wrapper configuration.

FIG. 17 is an exemplary package for the article of the present disclosure.

FIG. 18 is an exemplary configuration for a communication means of the present disclosure.

FIG. 19A is a perspective view of an absorbent capacity unit without the screen element for the Absorbent Capacity Test.

FIG. 19B is a perspective view of an absorbent capacity unit without the screen element for the Absorbent Capacity Test.

FIG. 19C is a perspective view of the screen element for the Absorbent Capacity Test.

Repeated use of reference characters in the present specification and drawings is intended to represent the same or analogous features or elements of the present invention. The drawings are representational and are not necessarily drawn to scale. Certain proportions thereof may be exaggerated, while others may be minimized.

Test Methods

Unless otherwise noted, all tests are performed at a temp of 23±2° C. and a relative humidity of 50±5%.

### Light Transmittance Test

Light transmittance (also referred to herein as light transmission) is measured consistent with ASTM D-1300 utilizing a GARDNER HAZE GUARD PLUS Model #4725 (available from BYK Gardner, having a place of business located in Columbia, Md., U.S.A.). In particular, a flat sample of the material to be tested is placed in the round holder having approximately a 60 mm diameter. Measurements are then taken by placing the flat sample in the measuring port. The haze port is used for measuring light transmittance. A series of five samples are measured and the average value of the five samples provides the light transmittance. Haze and clarity may also be measured using the GARDNER HAZE GUARD PLUS unit.

Color Measurement Test.

In some aspects of the present invention, hue, luminosity and saturation/vividness can be measured as described in the following paragraphs.

A Zeiss KS400 Image Analysis system is used for feature identification and colorimetric measurement. The Zeiss KS400 image analysis incorporates a Zeiss AxioCam color CCD camera (1300×1030 pixels, 3 channel color, 8 bit per channel) having a 20 mm AF-Nikkor lens (f/2.8). The camera is mounted vertically facing down onto a sample stage and has an effective field of view of 97 mm×80 mm Incident sample stage illumination is effected by four incandescent floodlamps (Sylvania) on a double Variac (70%; 90%), resulting in an illuminance of approximately 11,000 lux. The lamps above the left and right edges of the sample stage are directed towards the field of view at approximately 45 degrees.

The camera black reference is determined with the lens cap on. The camera white reference is determined with a Polaroid 803 positive with 15 ms exposure. To account for the warm color illumination bias of floodlamps, the red, green, and blue (RGB) values are offset using the white selection tool in the KS400 software, resulting in corrected RGB values that yield a white image.

Samples are placed on the stage (normal viewing angle) and under 0.64 mm plate glass to minimize topographical effects. Images of the color-bearing graphical portion are acquired at 15 ms exposure.

Image analysis is performed in Matlab (v.6.5.1, release 13; Mathworks, Inc) with the Image Processing Toolbox (v4.0). RGB images are converted to hue, saturation, and value (HSV) space using Matlab's hsv2rgb.m command. Choosing a saturation lower limit of 0.05 (0-1 scale) results in practical detection of all the colored/inked portions of the graphic. The hue, saturation, and value (i.e., luminosity) densiometric distributions are calculated for the detected regions in each image.

### Hunter Color Test

In some aspects of the present invention, hue, luminosity and saturation/vividness can be measured as described in the following paragraphs.

The color scale values of the graphic color of a sample specimen can be measured using the CIE LAB scale. Measurements are made with a Hunter Color reflectance meter. A complete technical description of the system can be found in an article by R. S. Hunter, ‘photoelectric color difference Meter’, Journal of the Optical Society of America, Vol. 48, pp. 985-95, 1958. Devices specially designed for the measurement of color on the Hunter scales are described in U.S. Pat. No. 3,003,388 to Hunter et al. Additional information can be found at www.hunterlab.com.

Colors can be measured according to an internationally recognized 3-D solid diagram of colors where all colors that are perceived by the human eye are converted into a numerical code. The CIE LAB system is similar to Hunter L, a, b and is based on three dimensions, specifically L*, a*, and b*.

When a color is defined according to this system, L* represents lightness (0=black, 100=white). A shiny white sample will yield an L* value near 100 while a dull black sample will yield an L* value of about 0. a* and b* independently each represent a two color axis, a* representing the axis red/green (+a=red,−a=green), while b* represents the axis yellow/blue (+b=yellow,−b=blue).

A color may be identified by a unique ΔE value (i.e., difference in color from some standard or reference), which is mathematically expressed by the equation:

ΔE*=[(L*_{X·}−L*_{Y})²+(a*_{X·}−a*_{Y})²+(b*_{X}−b*_{Y})²]^{1/2}

where “x” represents the standard or reference sample which may either be a “white” sample or a “colored” sample, e.g., one colored shade may be compared to another colored shade.

### Absorbent Capacity Test

A dry sample specimen is weighed at a temperature of 23±2° C. and a relative humidity of 50±5% on a scale capable of reading to 0.01 gram. This is the Dry Article Weight. The entire sample (body-facing surface up) is completely submerged in a sufficient amount of Menses Simulant A, at a temperature of 23±2° C. for 30 minutes, hereinafter referred to as the “saturation step.” Menses Simulant A is swine blood diluted to a hematocrit level of 30% by volume, with sheared, thick egg white added to mimic the mucin component of menses. Menses Simulant A is available from Cocalico Biologicals, Inc., a business having offices located in Reamstown, Pa., U.S.A., and is described in U.S. Pat. No. 5,883,231 to Achter et al., which is incorporated herein by reference in a manner that is consistent herewith. The sample is saturated in an appropriately sized tub that is placed on a rocker to keep the components of Menses Simulant A thoroughly mixed. The rocker is utilized to help provide an agitating rocking motion that prevents the components of Menses Simulant A from separating. Suitable rockers are well-known to those skilled in the art and are capable of keeping the menses simulant thoroughly mixed without changing other properties of the menses simulant. The sample should remain completely submerged in Menses Simulant A during the rocking motion of the saturation step.

After the 30-minute saturation time, the soaked sample is placed horizontally (body-facing surface down) on a two-layer screen of an absorbent capacity unit, and excess Menses Simulant A is allowed to drip from the sample for 5 minutes.

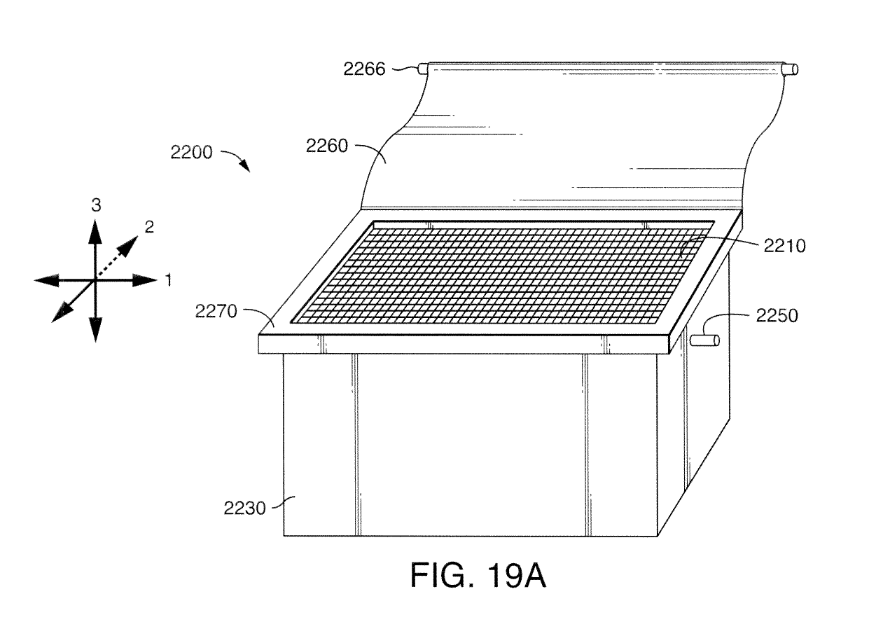

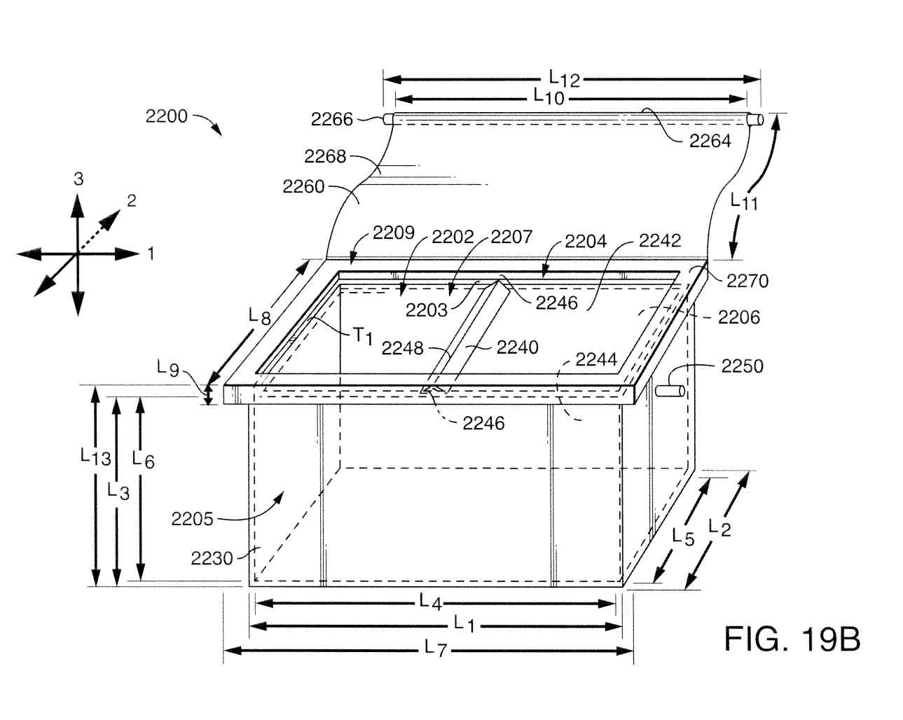

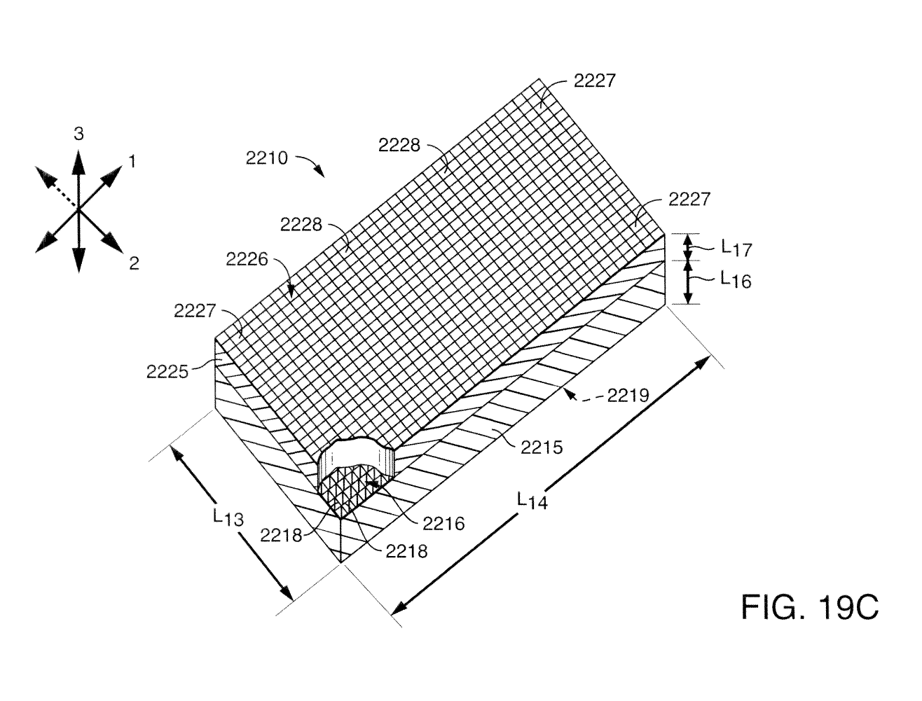

As seen in FIGS. 19A-19C, the absorbent capacity unit 2200 has a longitudinal direction 1 that defines length, and a transverse direction 2 that is perpendicular to the longitudinal direction 1 and defines width, where the longitudinal direction 1 and the transverse direction 2 form a plane (hereinafter the “x-y plane”). The unit 2200 also has a z-direction 3 that is perpendicular to the x-y plane and defines height. The absorbent capacity unit 2200 includes a hollow, rigid plastic box 2230 having an outer length L₁ of 399 mm, an outer width L₂ of 204 mm, an outer height L₃ of 180 mm, and a vertical wall thickness T₁ of about 10 mm. The box 2230 also includes a first opening 2202 at the top 2207 which defines a first inner volume having a length L₄ of 379 mm, a width L₅ of 184 mm and a height L₆ of 174 mm.

Located around the outside perimeter at the top portion of the box 2230 is a ridge frame 2270. The ridge frame 2270 forms a rectangular frame, having an outside length L₇ of 445 mm, an inside length equal to the outside length L₁ of the box 2230 of 399 mm, an outside width L₈ of 250 mm, an inside width equal to the outside width L₂ of the box 2230 of 204 mm, and a height L₉ of 22 mm. The ridge frame 2270 is adhesively secured to outside perimeter of the box 2230 such that approximately 11 mm of the height L₉ is positioned above the top edge 2203 of the box 2230. Thus, the total height L₁₃ of the absorbent capacity unit 2200 is 191 mm. Accordingly, the ridge frame 2270 defines a second opening 2204 located above the first opening 2202 and defines a second inner volume having a length of 399 mm, a width of 204 mm and a height of 11 mm.

The unit 2200 further includes a support brace 2240 having a length equal to the first inner volume width L₅ of 184 mm and is attached transversely to the back inner wall 2242 and front inner wall 2244 of the box 2230, and is located at the longitudinal center point 2246 of the walls 2242,2244. The support brace 2240 has a triangular profile and is positioned such that the top point of the triangular profile forms a transverse ridge 2248, where the ridge 2248 is adjacent to, and runs even with, the top edge 2203 of the box 2230.

The unit 2200 further includes a tubular vacuum inlet 2250 having an inside diameter of 0.64 mm to which a vacuum hose is attached and a vacuum is applied to the absorbent capacity unit 2200. In addition, the unit 2200 includes a mat 2260 comprising a flexible rubber substrate 2268, and having an approximate length L₁₀ of 450 mm, an approximate width L₁₁ of 374 mm and an approximate thickness of 0.5 mm as measured with a Mitutoyo Digimatic micrometer (available from Mitutoyo America Corp., having a place of business located in Aurora, Ill., U.S.A.). A first longitudinal side (not shown) of the mat 2260 is secured below ridge frame 2270 at the outer back wall 2206 of the unit 2200. A second longitudinal side 2264 that is distal and opposite of the first longitudinal side of the mat 2260 can be moved freely, thus forming a cover that can be lowered to completely envelop the top 2209 of the unit 2200. Disposed within the mat 2260 proximate the second longitudinal side 2264 is a wooden doweling 2266 having a length L₁₂ of 535 mm, a diameter of 16 mm and a weight of 63.3 grams. More specifically, the longitudinal edge portion (not shown) of the flexible rubber substrate 2268 is folded around the doweling 2266, and the edge portion (not shown) of the substrate 2268 is adhesively sealed to the substrate 2268 such that a length of the doweling 2266 equal to the length L₁₀ of the mat 2260 is completely enveloped by the substrate 2268. Accordingly, the second longitudinal side 2264 of the mat 2260 is considered to be located at the planar edge of the mat 2260 with the doweling 2266 in place, rather than the actual edge of the rubber substrate 2268. Thus, the width L₁₁ of the mat 2260 is measured from first longitudinal side to second longitudinal side 2264.

The absorbent capacity unit 2200 further includes a two-layer screen 2210, as seen in FIG. 19C. The two-layer screen 2210 comprises a bottom screen 2215 and a top screen 2225 that are substantially coextensive. The screens 2215,2225 each have a length L₁₄ of approximately 398 mm and a width L₁₄ of approximately 202 mm. The bottom screen has a height L₁₆ of 10 mm, and is in the form of a grating. More specifically, bottom screen 2215 has a series of square holes 2216 having a length and width of 15 mm×15 mm each, which extend the entire z-direction 3 of the bottom screen 2215 (i.e., 10 mm). Each hole 2216 is defined by, and separated by, a plastic wall 2218 having a height of 10 mm and a thickness of 1.5 mm. The top screen 2225 has a height (thickness) L₁₇ in the z-direction 3 of approximately 1 mm. The top screen 2255 consists of a series of woven strands 2228 extending in the longitudinal direction 1 and the transverse direction 2, defining square holes 2226 having a length and width of 4 mm×4 mm, which extend the entire z-direction 3 of the top screen 2225 (i.e., 1 mm). The top screen 2225 is attached to the bottom screen 2215 via tie wraps 2227 located near the four corners of the two-layer screen 2210. The two-layer screen 2215 is configured to fit into the second opening 2204 of the absorbent capacity unit 2200 and the bottom surface 2219 rests on the top edge 2203 of the box 2230, as well as on the transverse ridge 2248 of the support structure 2240.

After the sample has dripped for 5 minutes, the saturated sample is carefully removed from the absorbent capacity unit 2200 and subsequently weighed to give the Saturated Article Weight. The weight of Menses Simulant A held in the sample is the Saturation Capacity of the article, as calculated by the formula:

$$Saturation Capacity = Saturated Article Wt . - Dry Article Wt . ;$$

$$and$$

$$Specific Total Saturation Capacity = \frac{( \begin{matrix}Saturated Article Wt . - \\ Dry Article Wt .\end{matrix} )}{( Dry Article Wt . )}$$

After the saturated sample has been weighed, the sample is once again placed horizontally onto the two-layer screen 2210 (body-facing surface down), and the mat 2260 lowered over the sample such that the entire top side 2209 of the unit 2200 is enveloped by the mat 2260, with the doweling 2266 and the second end 2264 of the mat 2260 hanging freely below the ridge frame 2270 at the front side 2205 of the unit 2200. A negative vacuum is applied to to the unit 2200 through the tubular vacuum inlet 2250 to deliver a −0.5 psi pressure (−3.45 kpa) to the sample, such that the vacuum is applied evenly across body-facing surface of the sample. The vacuum is applied for 5 minutes during which time Menses Simulant A is allowed to drip through the two-layer screen 2210. The sample becomes somewhat compressed during this “vacuum step.” After the 5 minutes is complete, the rubber mat is lifted off of the unit 2200, and the compressed sample is carefully removed and weighed to give the Compressed Article Weight. The weight of the liquid remaining in the compressed sample after the vacuum step is the Retention Capacity. The Specific Retention Capacity is the weight of the liquid remaining in the compressed article divided by the Dry Article Weight. Accordingly:

$$Retention Capacity = ( \begin{matrix}Compressed Article Wt . - \\ Dry Article Wt .\end{matrix} ) ;$$

$$and$$

$$Specific Total Retention Capacity = \frac{( \begin{matrix}Compressed Article Wt . - \\ Dry Article Wt .\end{matrix} )}{( Dry Article Wt . )}$$

### DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As used herein, the term “absorbent product” or “absorbent article” refers to any article used to control bodily fluids that are configured to absorb and retain bodily exudates, including urine, blood, menses, and other bodily discharges, such as sweat and vaginal secretions resulting from sexual activity and the like. In addition, the term is intended to include odor absorbing articles.

As used herein, the term “attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements.

As used herein, “body-facing” means that surface or side which is intended to be disposed toward or placed adjacent to the body of the wearer during ordinary use.

The term “garment-facing” means that surface or side that is on the opposite side from the body-facing surface or side. The garment-facing surface is an outward surface and is intended to be disposed to face away from the wearer's body during ordinary use. The garment-facing surface is generally arranged to face toward or placed adjacent to the wearer's undergarments or clothing when an absorbent article is worn.

The term “bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements.

The term “coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a foraminous belt. The forming surface may include a gas-pervious material that has been placed onto the forming surface.

Exemplary coform processes are described in U.S. Pat. Nos. 4,100,324 to Anderson et al.; 4,587,154 to Hotchkiss et al.; 4,604,313 to McFarland et al.; 4,655,757 to McFarland et al.; 4,724,114 to McFarland et al.; 4,100,324 to Anderson et al.; and U.K. Patent GB 2,151,272 to Minto et al., each of which is incorporated herein by reference in a manner that is consistent herewith.

As used herein, the term “color” and its derivatives is intended to mean an individual's perception of the spectral composition of visible light coming from a portion of an object. Color characteristics include hue, saturation and luminosity. Each is a separate color characteristic. Hue is the attribute of a color which allows it to be classified as a given color. Saturation, which is sometimes referred to as vividness or chroma, is the intensity of the color. Saturation is the degree of freedom from gray. Moreover, luminosity, sometimes referred to as value, is the degree of lightness (paleness) or darkness in a color and is measured from white to black. A measurement of hue, saturation and luminosity are described in more detail herein. The term “complex liquid” describes a liquid generally characterized as being a viscoelastic liquid pertaining primarily to aqueous liquids associated with menstruation.

As used herein, the term “connected” is intended to mean directly connected and indirectly connected. By directly connected, it is intended that the connected elements are in contact with one another or affixed to one another. By indirectly connected, it is intended that one or more intervening or intermediate elements are between the two elements which are secured or “connected” together. The intervening elements may be affixed.

As used herein, the term “continuous” means marked by uninterrupted extension in space, time, or sequence.

The terms “current absorbent product” or “current absorbent product choice” are used herein to describe an absorbent product (or products) that is currently being used or purchased by a consumer.

As used herein, the term “design” means a collection of points, lines, line segments, curves, or areas that were put together according to a plan.

As used herein, the term “disposable” is used herein to describe personal care articles that are not intended to be laundered or otherwise restored or reused as a personal care article after a single use.

As used herein, the term “feminine care pads” includes pads, liners, including but not limited to ultrathin, feminine napkins, maxi-pads, overnights, and the like.

As used herein, the term “free-form” means irregular or asymmetric.

As used herein, the term “fun” means that which provides enjoyment, entertainment, or amusement; playful.

The terms “future absorbent product” or “future absorbent product choice” are used herein to describe an absorbent product (or products) that is not currently being used or purchased by a consumer but that a consumer may potentially transition into (e.g., a product which may potentially be used or purchased by a consumer at some future time).

The term “hydrophilic” describes materials which are wetted by aqueous liquids in contact with the materials. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, materials having contact angles less than 90 degrees are designated “wettable” or hydrophilic, and fibers having contact angles greater than 90 degrees are designated “nonwettable” or “hydrophobic”.

As used herein, the term “personal care absorbent article” includes, but is not limited to, articles such as diapers, diaper pants, baby wipes, training pants, absorbent underpants, child care pants, swimwear, and other disposable garments; feminine care products including sanitary napkins, wipes, feminine pads, menstrual pants, pantiliners, panty shields, interlabials, tampons, and tampon applicators; adult-care products including wipes, pads such as breast pads, containers, incontinence products, and urinary shields; clothing components; bibs; athletic and recreation products; and the like.

As used herein, the term “related”, and its derivatives, is used to describe the way or degree two items fit together. For example, two items are considered related if some aspects of one of the items are identical to similar aspects of another item, or are a part of the same brand or product line. For example, pantiliners, ultrathin pads, maxi-pads, and overnight pads are all considered to be related.

The terms “spunbond” and “spunbonded fiber” refer to fibers which are formed by extruding filaments of molten thermoplastic material from a plurality of fine, usually circular, capillaries of a spinneret, and then rapidly reducing the diameter of the extruded filaments.

As used herein, the term “superabsorbent material” refers to a water-swellable, generally water-insoluble, hydrogel-forming polymeric absorbent material, which is capable of absorbing at least about 10, suitably about 30, and possibly about 60 times or more its weight in physiological saline (e.g., saline with 0.9 wt % NaCl). In contrast, the term “absorbent material” refers to a material that is capable of absorbing at least about 5 times or more its weight in physiological saline (e.g., saline with 0.9 wt % NaCl). In the case of complex fluids, the term “superabsorbent material” refers to a water-swellable, generally water-insoluble, hydrogel-forming polymeric absorbent material, which is capable of absorbing at least about 10 times or more its weight in menses simulant (i.e., menses simulant composed of swine blood diluted with swine plasma to provide a hematocrit level of 35% (by volume), suitably HEMATOSTAT-2, available from Separation Technology, Inc., a business having offices located in Altamonte Springs, Fla., U.S.A.).

As used herein, the term “visible” or “visually perceived” is intended to mean an attribute or feature which a human user or consumer can visually discern (e.g., the color or the pattern) with the unaided eye (excepting standard corrective lenses adapted to compensate for near-sightedness, farsightedness, stigmatism, or other corrected vision) in lighting at least equal to the illumination of a standard 100 watt incandescent white light bulb. Generally for a consumer or user, the attribute should be visible in the range of about 0.25 feet (0.075 meters) to about 3 feet (0.91 meters). As used herein, “discern,” “perceived” or “perception” is the ability to recognize an attribute or feature when the visual angle that the attribute or feature subtends is greater than about 5 minutes of visual arc and less than about 45 minutes of visual arc as determined by the following equation:

Minutes of visual arc=3438*(length of the object/distance from object)

### Where:

Length of the object=size of the object measured perpendicular to the line of sight; and
Distance from object=distance from the front of the eye to the object along the line of sight
   A minute of visual arc is 1/60th of 1 degree.

These terms may be defined with additional language in the remaining portions of the specification.

### DETAILED DESCRIPTION

The invention provides a system for visually identifying the performance level of disposable personal care absorbent articles, as well as a method for providing such. In this particular aspect, the system includes a first disposable personal care absorbent article and at least a second disposable personal care absorbent article, each having a body-facing surface and a garment-facing surface. In some aspects, the invention also provides disposable personal care absorbent articles having unique properties, such as graphic patterns that are bold, brilliant and/or vivid (i.e., not discreet), and can in some aspects further evoke a culture of “fun,” as well as a method for providing such. These unique properties are non-traditional, or even taboo, and teach away from current articles that continue to promote the current culture of sensitivity, discreteness and embarrassment felt by the users of such articles. Further discussion of these features, as well as other features of the present invention, can be found in Provisionally filed U.S. Patent Application 61/206,459 to Loyd, filed Jan. 30, 2009, which is incorporated herein by reference in a manner that is consistent herewith.

In some aspects, the system also includes a performance identification device comprising a pattern (e.g., a printed pattern) that is visible to a user on at least one of the body-facing surface or the garment-facing surface of the articles. In some desirable aspects, the pattern is continuous in nature and/or in free-form. In some aspects, the first disposable personal care absorbent article and the second disposable personal care absorbent article are related. In further aspects, the second disposable personal care absorbent article has a higher performance value than the first disposable personal care absorbent article. In additional aspects, such as in the case of printed patterns, the pattern increases in surface area, ink coverage and/or intensity as the performance value also increases. Furthermore, in some desirable aspects, the pattern of the second disposable personal care absorbent article is thematically related or visually coordinated with the pattern of the first disposable personal care absorbent article.

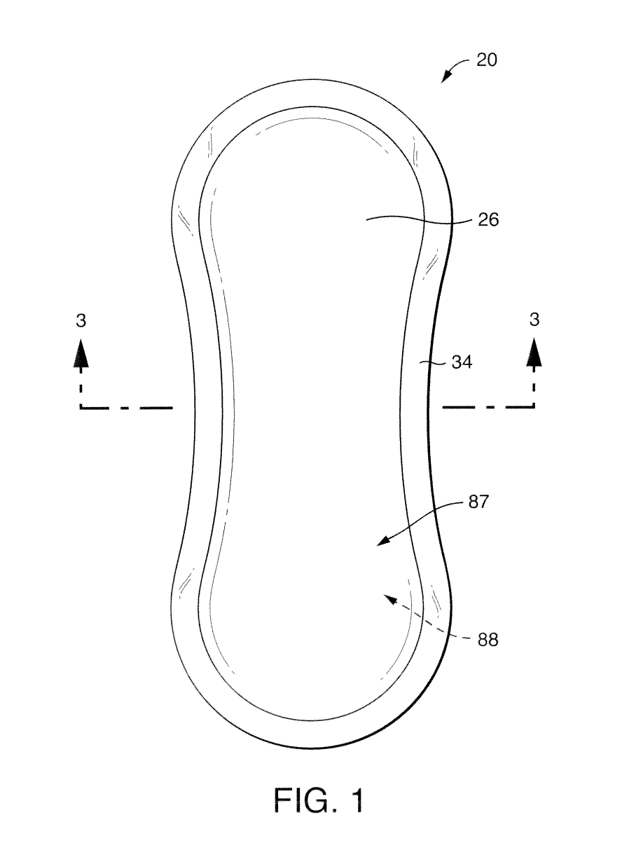

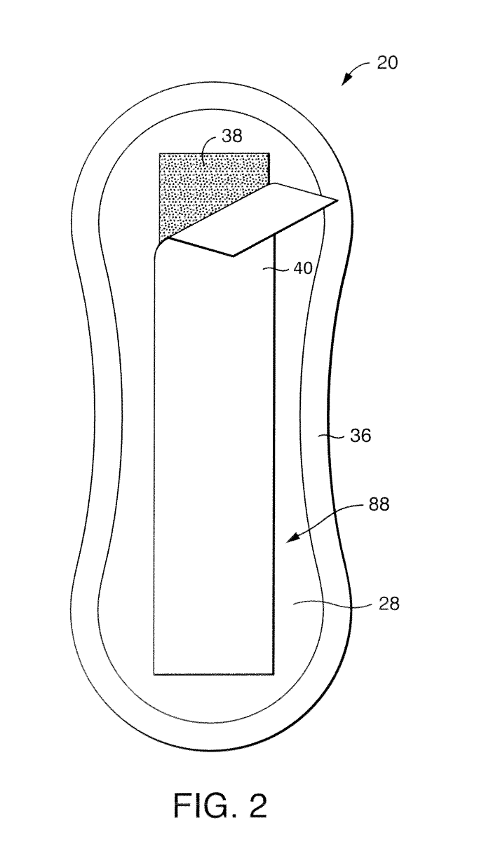

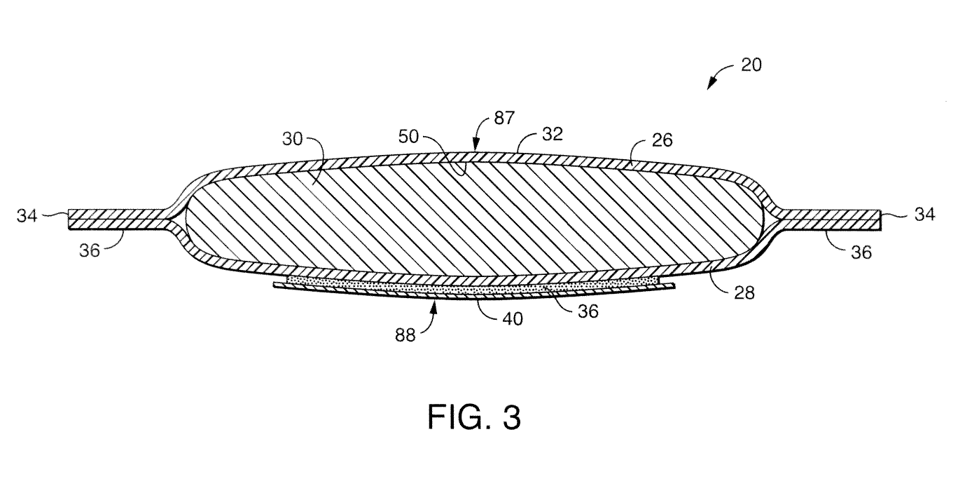

To gain a better understanding of the present invention, attention is directed to FIGS. 1-6 for exemplary purposes showing a feminine pad. It is understood that the present invention is suitable for use with various other absorbent articles, including but not limited to other personal care absorbent articles as defined herein, without departing from the scope of the present invention.

Referring to the drawings and in particular to FIGS. 1-6, a representative article, such as the representatively shown feminine care article, is shown. The feminine care article can, for example, be a feminine care pad, which is indicated generally at 20. As illustrated in FIGS. 1-3, the pad 20 can include a topsheet 26, a backsheet 28, and/or an absorbent core 30 positioned between the topsheet and the backsheet. The topsheet 26 may include a layer constructed of any operative material, and may be a composite material. For example, the topsheet 26 can include a woven fabric, a nonwoven fabric, a polymer film, a film-fabric laminate, or the like, as well as combinations thereof. Examples of a nonwoven fabric include spunbond fabric, meltblown fabric, coform fabric, a carded web, a bonded-carded-web, a bicomponent spunbond fabric, or the like, as well as combinations thereof.

For example, the topsheet 26, if present, can include a woven fabric, a nonwoven fabric, a polymeric film that has been configured to be operatively liquid-permeable, or the like, as well as combinations thereof. Other examples of suitable materials for constructing the topsheet 26 can include rayon, bonded-carded-webs of polyester, polypropylene, polyethylene, nylon, or other heat-bondable fibers, polyolefins, such as copolymers of polypropylene and polyethylene, linear low-density polyethylene, aliphatic esters such as polylactic acid, finely perforated film webs, net materials, and the like, as well as combinations thereof.

A more particular example of a suitable topsheet 26 material can include a bonded-carded-web composed of polypropylene and polyethylene, such as has been used as a cover stock for KOTEX brand pantiliners, and has been obtainable from Vliesstoffwerk Christian Heinrich Sandler GmbH & Co. KG, a business having an address at Postfach 1144, D95120 Schwarzenbach/Saale, Germany. Other examples of suitable materials are composite materials of a polymer and a nonwoven fabric material. The composite materials are typically in the form of integral sheets generally formed by the extrusion of a polymer onto a web of spunbond material. As used herein, the terms “spunbond” or “spunbonded fiber” refer to fibers which are formed by extruding filaments of molten thermoplastic material from a plurality of fine, usually circular, capillaries of a spinneret, and then rapidly reducing the diameter of the extruded filaments.

In a desired arrangement, the topsheet 26 can be configured to be operatively liquid-permeable with regard to the liquids that the pad 20 is intended to absorb or otherwise handle. The operative liquid-permeability may, for example, be provided by a plurality of pores, perforations, apertures or other openings, as well as combinations thereof, that are present or formed in the topsheet. The apertures or other openings can help increase the rate at which bodily liquids can move through the thickness of the topsheet 26 and penetrate into the other components of the pad 20 (e.g., the absorbent core 30). The selected arrangement of liquid-permeability is desirably present at least on an operative portion of the topsheet 26 that is appointed for placement on the body-side of the pad 20.

The topsheet 26 can provide comfort and conformability, and can function to direct bodily exudates away from the body and toward the absorbent core 30. In a desired feature, the topsheet 26 can be configured to retain little or no liquid in its structure, and can be configured to provide a relatively comfortable and non-irritating body-facing surface 32 (broadly, an “engagement surface”) next to the skin of the wearer. The topsheet 26 can be constructed of any material easily penetrated by bodily fluids that contact the surface of the topsheet.

The topsheet 26 can also have at least a portion of its body-facing surface 32 treated with a surfactant to render the topsheet more hydrophilic. The surfactant can permit arriving bodily liquids to more readily penetrate the topsheet 26. The surfactant may also diminish the likelihood that the arriving bodily fluids, such as urine or complex liquid, will flow off the topsheet 26 rather than penetrate through the topsheet into other components of the pad 20 (e.g., the absorbent core 30). In a particular configuration, the surfactant can be substantially evenly distributed across at least a portion of the upper, body-facing surface 32 of the topsheet 26 that overlays the upper, body-side surface of the absorbent core 30.

The topsheet 26 may be maintained in secured relation with the absorbent core 30 by bonding all or a portion of the adjacent surfaces to one another. A variety of bonding articles known to one of skill in the art may be utilized to achieve any such secured relation. Examples of such articles include, but are not limited to, the application of adhesives in a variety of patterns between the two adjoining surfaces, entangling at least portions of the adjacent surface of the absorbent with portions of the adjacent surface of the topsheet 26, or fusing at least portions of the adjacent surface of the topsheet to portions of the adjacent surface of the absorbent core 30.

The topsheet 26 typically extends over the upper, body-facing surface 32 of the absorbent core 30, but can alternatively extend around the pad 20 to partially or entirely surround or enclose the absorbent core 30. Alternatively, the topsheet 26 and the backsheet 28 can have peripheral margins 34,36 that extend outwardly beyond the terminal, peripheral edges of the absorbent core 30, and the extending margins can be joined together to partially or entirely surround or enclose the absorbent core.

In some aspects, the topsheet 26 can be in the form of a single cover. In other aspects, the topsheet can be in the form of a dual cover. In still other aspects, the topsheet can be in the form of a three-piece cover, or more. For example, one portion of the topsheet can be a film or foam, which may be perforated, and another portion of the topsheet can be a nonwoven.

The backsheet 28, if present, may include a layer constructed of any operative material, and may or may not have a selected level of liquid-permeability or liquid-impermeability, as desired. In a particular configuration, the backsheet 28 may be configured to provide an operatively liquid-impermeable backsheet structure. The backsheet 28 may, for example, include a polymeric film, a woven fabric, a nonwoven fabric, or the like, as well as combinations or composites thereof. For example, the backsheet 28 may include a polymer film laminated to a woven or nonwoven fabric. In a particular feature, the polymer film can be composed of polyethylene, polypropylene, polyester, or the like, as well as combinations thereof. Additionally, the polymer film may be micro-embossed, have a printed design, have a printed message to the consumer, and/or may be at least partially colored. Desirably, the backsheet 28 can operatively permit a sufficient passage of air and moisture vapor out of the pad 20, particularly out of an absorbent core 30, while blocking the passage of bodily liquids. An example of a suitable backsheet 28 can include a breathable, microporous film, such as a HANJIN Breathable Baffle available from Hanjin Printing, Hanjin P&C Company Limited, a business having offices located in Sahvonli.Jungan-mvu.Kongiu-City, Chung cheong nam-do, Republic of South Korea. This particular backsheet 28 material is a breathable film, which is white in color, dimple embossed and contains: 47.78% calcium carbonate, 2.22% TiO2, and 50% polyethylene.

Bicomponent films or other multi-component films can also be used as backsheet 28 material, as well as woven and/or nonwoven fabrics which have been treated to render them operatively liquid-impermeable. Another suitable backsheet 28 material can include closed cell polyolefin foam. For example, closed cell polyethylene foam may be employed. Still another example of a backsheet 28 material would be a material that is similar to a polyethylene film which is used on commercially sold KOTEX brand pantiliners, and is obtainable from Pliant Corporation, a business having offices located in Schaumburg, Ill., U.S.A.

The structure of the absorbent core 30 can be operatively configured to provide a desired level of absorbency or storage capacity. More particularly, the absorbent body can be configured to hold a liquid, such as urine, feces, menses, other complex liquid, or the like, as well as combinations thereof. The absorbent core 30 can include a matrix of absorbent fibers and/or absorbent particulate material, and the absorbent fiber can include natural and/or synthetic fiber. Additionally, the absorbent core 30 may include one or more components that can modify menses or menstrual liquid.

The absorbent core 30 can also include superabsorbent material. Superabsorbent materials are known to those skilled in the art, and may be in any operative form, such as particulate form. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof.

The hydrogel-forming polymers are preferably lightly crosslinked to render the material substantially water insoluble. Crosslinking may, for example, be by irradiation or covalent, ionic, Van der Waals, or hydrogen bonding. Suitable materials are available from various commercial vendors such as BASF and Evonik-Stockhausen, Inc. The superabsorbent material may desirably be included in an appointed storage or retention portion of the absorbent core, and may optionally be employed in other components or portions of the pad 20.

The absorbent core 30 can be arranged in any operative shape and/or design. For example, the absorbent core 30 may comprise a composite structure (not shown) having a selected plurality of strata or layers or a unitary structure. Moreover, the material of the absorbent core 30 can be selected and configured to provide desired liquid-intake properties to quickly absorb and pull liquid away from the body. Accordingly, the absorbent core 30 can provide the function of liquid intake and can also provide the functions of liquid distribution, spreading, liquid retention, and shape maintenance. The absorbent core 30 may include natural fibers, synthetic fibers, superabsorbent materials, a woven fabric; a nonwoven fabric; a wet-laid fibrous web; a substantially unbonded airlaid fibrous web; an operatively bonded, stabilized-airlaid fibrous web; or the like, as well as combinations thereof. Additionally, the absorbent core 30 may include one or more components that can modify menses or intermenstrual liquid.

In a particular arrangement, the absorbent core 30 can be a thermally-bonded, stabilized airlaid fibrous web available from Concert Fabrication (Concert code 225.1021), a business having offices located in Gatineaux, Quebec, Canada (e.g. Concert code 225.1021). The absorbent core 30 may also be a similar, stabilized airlaid fibrous web available from Buckeye Technologies, Inc., a business having offices located in Memphis, Tenn., U.S.A.

Additionally, a selected configuration of garment adhesive 38, such as the illustrated strip regions, may be distributed onto the garment-side surface of the pad 20 to help secure the napkin to the undergarment (not shown). Typically, the garment adhesive 38 can be distributed over the garment-side surface of the backsheet 28, and one or more layers or sheets of release material 40 can be removably placed over the garment adhesive 38 during storage prior to use.

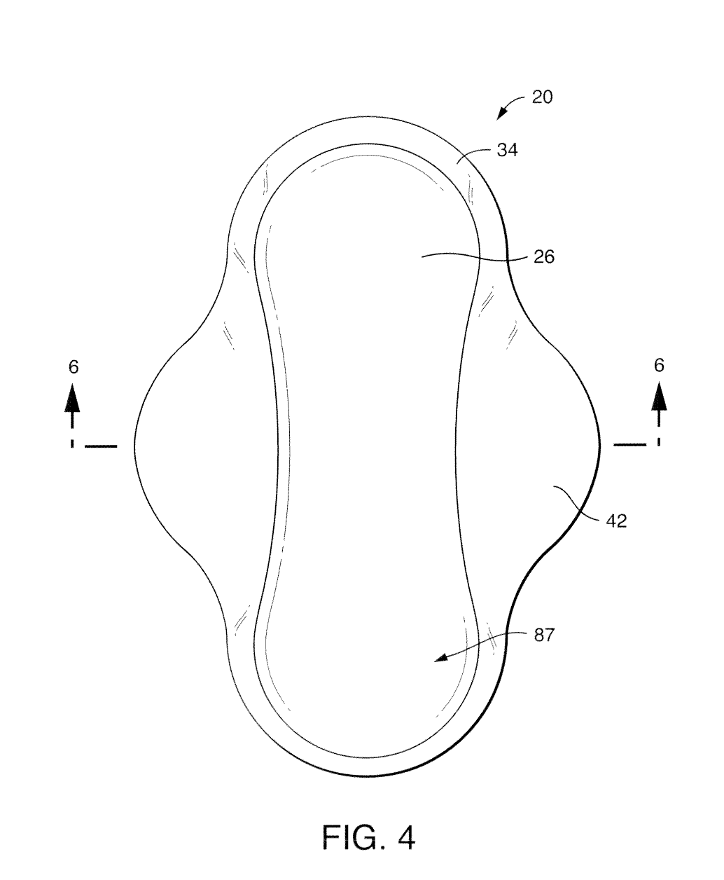

As illustrated in FIGS. 4-6, the pad 20 can include a system of wing portions 42 which can be integrally connected to appointed sections of the napkin. After placing the pad 20 in the undergarment, the wings 42 can be operatively wrapped and secured around the side edges of the undergarment to help hold the napkin in place as is known in the art. The wing portions 42 can be separately provided members that are subsequently attached or otherwise operatively joined to intermediate portions of the pad 20.

In other configurations, the wing portions 42 can be unitarily formed with one or more components of the pad 20. Either or both wing portions 42 may be formed from a corresponding, operative extension of the material employed to form the topsheet 26. Alternatively, either or both wing portions 42 may be formed from a corresponding, operative extension of the material employed to form the backsheet 28, or formed from a corresponding, operative combination of the topsheet 26 and backsheet materials.

The wing portions 42 can have any operative construction, and can include a layer of any operative material. Additionally, each wing portion 42 can comprise a composite material. For example, the wing portions 42 may include a spunbond fabric material, a bi-component spunbond material, a necked spunbond material, a neck-stretched-bonded-laminate (NBL) material, a meltblown fabric material, a bonded carded web, a thermal bonded carded web, a through-air bonded carded web, or the like, as well as combinations thereof.

Each wing portion 42 can be joined to its corresponding side region of the pad 20 in any operative manner. For example, the wing portion can be joined to the topsheet 26, the backsheet 28 or another pad 20 component, as well as any combination thereof. The wing portion 42 can be attached with hot melt adhesive, but any other operative adhesive or attachment mechanism may alternatively be employed.

In another feature, each wing portion 42, or any desired combination of the employed wing portion, can include a panel-fastener component 44 which is operatively joined to an appointed engagement surface of its associated wing. The panel-fastener can be configured to operatively attach to the wearer's undergarment and/or to any appointed, landing-zone portion of the pad 20. For example, the panel-fastener 44 can include a system of interengaging mechanical fasteners, a system of adhesive fasteners, a system of cohesive fasteners, or the like, as well as combinations thereof.

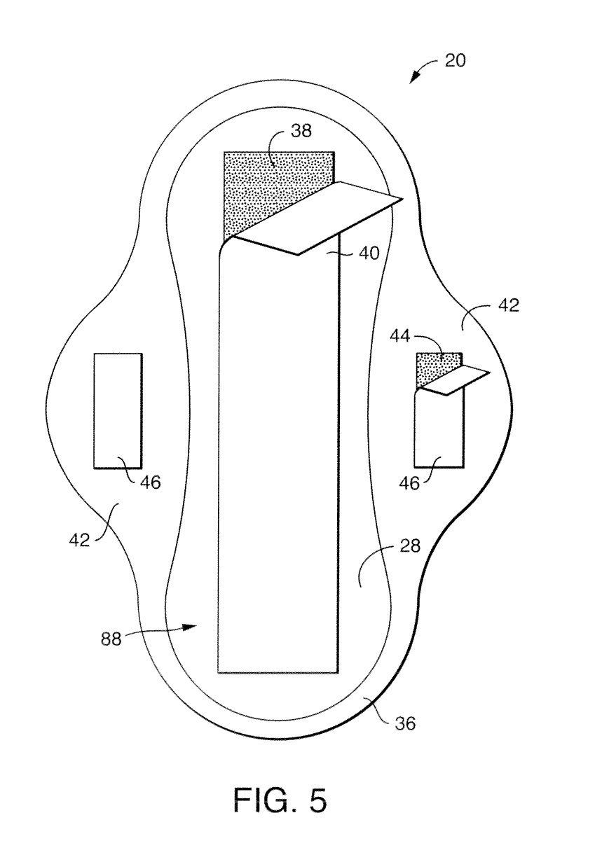

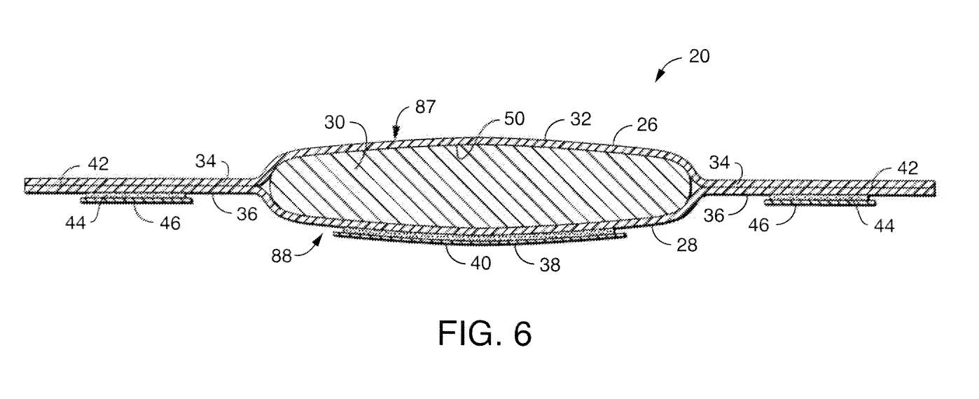

With reference to FIGS. 5 and 6, for example, either or both wing portions 42 can include a panel-fastener system 44 that alternatively incorporates an operative garment adhesive. The garment adhesive may be a solvent-base adhesive, a hotmelt adhesive, a pressure-sensitive adhesive, or the like, as well as combinations thereof. Each section of garment adhesive may be covered with a removable release material 46.

In the construction of the pad 20, the various components (e.g., topsheet 26, backsheet 28, absorbent core 30, wing portions 42) may be assembled and held together with any operative securement mechanism or system. For example, the desired attachments or securements can include adhesive bonds, cohesive bonds, thermal bonds, ultrasonic bonds, pins, snaps, staples, rivets, stitches, welds, zippers, or the like, as well as combinations thereof.

In some aspects, the absorbent articles of the present invention may optionally be folded and/or individually wrapped in various ways. For example, the absorbent articles may be folded and placed in an individual envelope or wrapper, or in other operative individual packaging. In a particular aspect, the removable release material of each article can be configured to provide a wrapping layer for an individual article, and the wrapping layer can extend across at least two major surfaces of its corresponding absorbent article and can at least partially enclose the individual absorbent article. Accordingly, an individual absorbent article can have a removable release material which includes its corresponding, individual wrapper material.

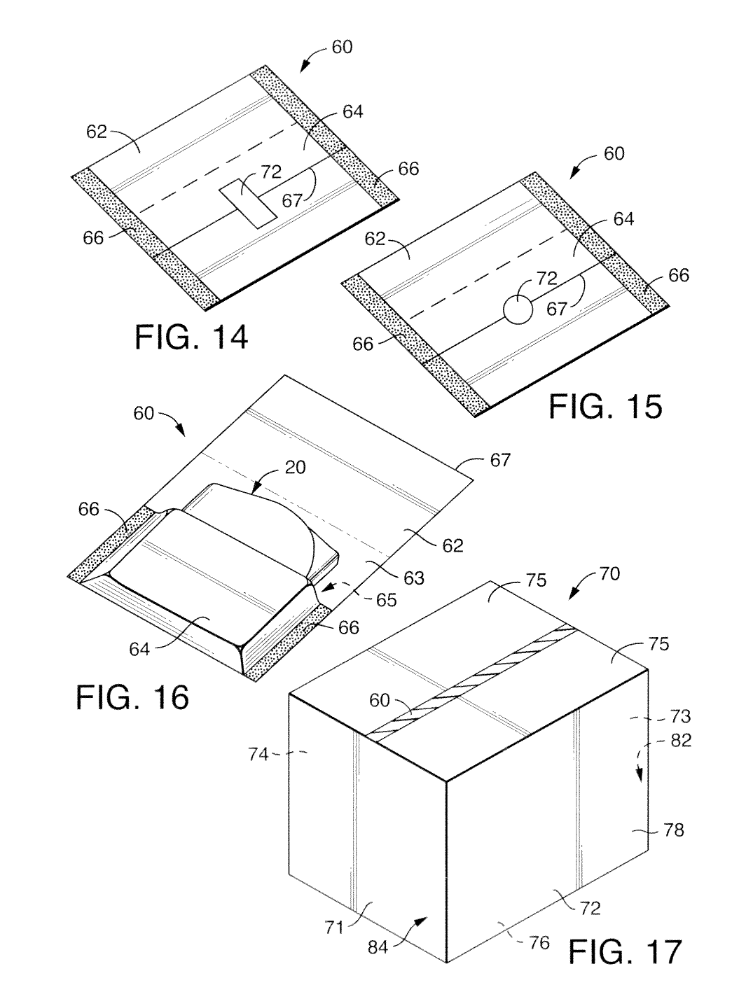

With reference to FIGS. 14-16, in other aspects, a wrapper 60 generally may be formed from a separate piece of wrapper material 62 using any methods known in the art. For example, the wrapper 60 may be formed by sealing together various portions of a wrapper material 62 in order to form an individual envelope or pouch that may encase the absorbent article 20. The wrapper 60 generally comprises an interior side 63 and an exterior side 64, with the interior side 63 facing the absorbent article 20 and forming a hollow interior 65 for receiving the absorbent article 20. The wrapper 60 may be sealed by heat-sealing, by crimp sealing, and/or by using a suitable adhesive, among other means to form a wrapper seal 66. Such methods of sealing are commonly known in the art. In some aspects, the wrapper 60 may optionally comprise opening aids 72, such as grasping means in the form of flaps, fins, tear strips, or the like, which increase ease of opening of the wrapper 60 by improving accessibility of an end edge 67 of the wrapper material 62. In this instance, the wrapper 60 is generally opened by pulling on a free edge of the wrapper material 62, which breaks the sealing of the wrapper (e.g., breaks an adhesively sealed wrapper), thus allowing the absorbent article 20 to be removed from the wrapper 60. Further discussion of the wrapper 60, as well as other features of the present invention, can be found in U.S. Patent Publication No. 2007/0142795 to Cohen et al., which is incorporated herein by reference in a manner that is consistent herewith.

In some aspects, the wrapper 60 may be formed from a material that is sufficiently transparent to allow an operative viewing of the article by the user through the wrapping material 62. By “transparent” it is meant that the material has a minimum light transmittance of about 45% or more, such as about 60% or more, or 80% or more, such as in the range of about 60% to about 79%, as measured by the Light Transmittance Test. The wrapper 60 can comprise any operative material. For example, the wrapping material 62 can comprise a polymer film, a woven fabric, a nonwoven fabric, a composite laminate or the like, and combinations thereof.

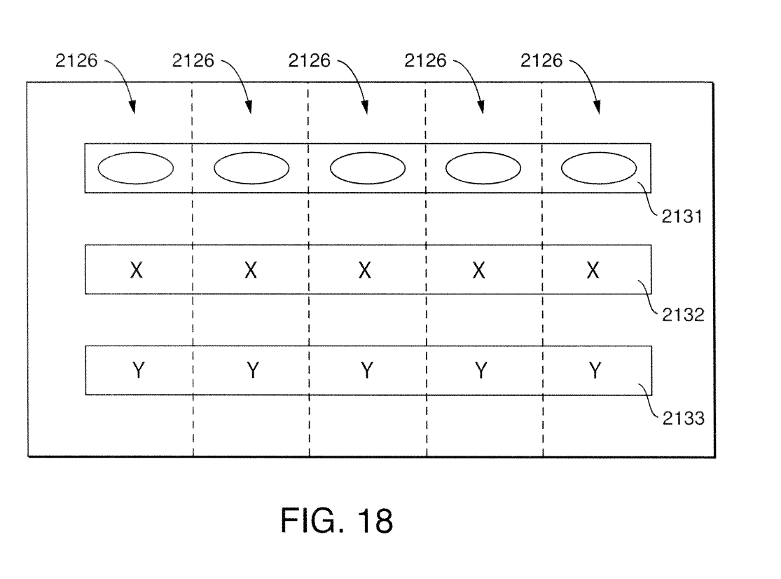

With reference to FIG. 17, in some aspects, the absorbent articles of the present disclosure may also be grouped together with several other related absorbent articles to form a package 70 of absorbent articles having a viewable surface area. If the package 70 has the general shape of a rectangular parallelepiped such as illustrated in FIG. 17 (i.e., four rectangular side panels 71,72,73,74, a rectangular top panel 75, and a rectangular bottom panel 76), then “viewable surface area” means the sum of the six areas corresponding to each of the six viewable rectangles or panels 71,72,73,74,75,76 that make up the rectangular parallelepiped.

The package 70 may comprise a plurality of the absorbent article 20, preferably two or more individual absorbent articles, and more preferably comprise eight, ten, or more preferably twenty absorbent articles per package. The absorbent articles that are packaged together may be individually wrapped, as discussed above, or optionally may be packaged unwrapped. In some aspects, the package 70 of absorbent articles comprises a plurality of the same absorbent article (e.g., a package of pads all having the same performance value). Alternatively, in some desirable aspects, the package 70 of absorbent articles may comprise more than one type of related absorbent article packaged together (e.g., a package comprising pads having different performance levels and/or performance identification device). For example, it may be desirable to package a first feminine care pad and a second feminine care pad into the same package 70, each having a different performance value (e.g., absorbent capacity).

The package 70 can comprise a soft or hard packaging material. Soft packaging includes flexible envelopes and packages made of sheet plastic and/or paper. Hard packaging includes generally less flexible packages made of plastic (e.g., tubs and buckets), metal or paperboard, and combinations thereof. Examples of suitable packaging materials include, for example, paper, cardboards, polymeric materials, coated paper, composite structures, or any other suitable packaging material. The package 70 can have any operative configuration. For example, the package 70 may comprise a bag, a carton, a container, a box, or the like, as well as combinations thereof. The various panels of the package 70 can be flexible panels, rigid panels, semi-rigid panels, or the like, as well as combinations thereof. As discussed above with respect to wrappers 60, the package 70 may also be formed by sealing together various portions of a packaging material 78 in order to form a package 70 encasing the absorbent articles 20. The package 70 generally comprises an interior side 82 and an exterior side 84, with the interior side 82 facing the absorbent articles 20 and forming a hollow interior for receiving the absorbent articles 20. In certain embodiments, the package 70 may comprise an opening mechanism (not shown), which provides access to the absorbent articles 20. Such opening mechanisms include, for example, a perforated section of packaging, a grasping means in the form of flaps, fins, tear strips, or the like, which increase ease of opening of the package 70 by improving accessibility of an end edge of the package material, and combinations thereof.

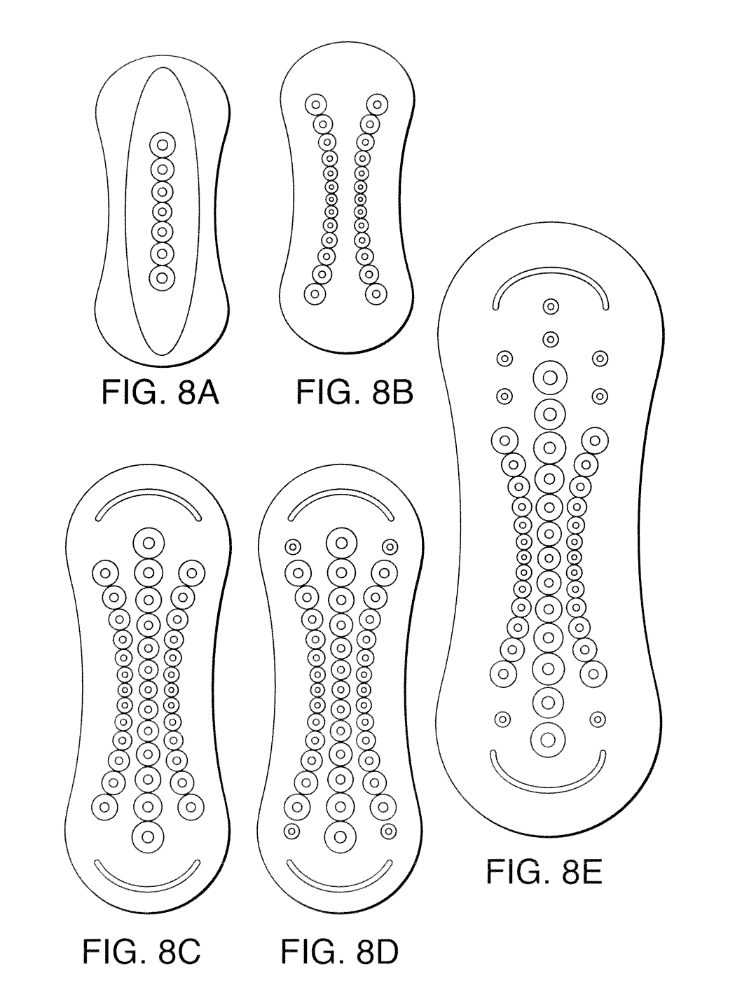

The invention provides a system and method for visually identifying the performance level (e.g., absorbency, softness, intake rate, and the like) of two or more disposable personal care absorbent articles. For example, the disposable personal care absorbent article 20 has a body-facing surface 87 and a garment-facing surface 88. The article 20 can include a performance identification device comprising a pattern, such as a printed pattern, or a printed pattern and embossing, that is visible to a user on at least one of the body-facing surface 87 or the garment-facing surface 88 of the article 20. The pattern increases in total surface area as the performance value (i.e., level) also increases, as compared to another related article. As used herein, the term “total surface area” refers to the amount of ink and/or embossing utilized to form the pattern. To illustrate, FIGS. 8A-10E show arrays of related absorbent articles comprising the performance identification device of the present invention. In the illustrated embodiments, a printed pattern on the body-facing surface of each article is visually perceived by a user. More particularly, FIGS. 8A-10E show arrays (i.e., FIGS. 8A-8E, FIGS. 9A-9E and FIGS. 10A-10E) of related feminine care pads, with each pad in the respective array having a different absorbency level, such as saturated capacity and/or retention capacity, as measured by the Absorbent Capacity Test. FIGS. 8A, 9A and 10A show an invisible-feel pantiliner having the lowest absorbency level of the respective array; FIGS. 8B, 9B and 10B show a body-contour liner having a higher absorbency level than the corresponding invisible-feel liner of FIGS. 8A, 9A and 10A; FIGS. 8C, 9C, and 10C show an ultrathin feminine pad having a higher absorbency level than the corresponding body-contour liner of FIGS. 8B, 9B and 10B; FIGS. 8D, 9D and 10D show a heavy flow maxi-pad having an even higher absorbency level than the corresponding ultrathin pad of FIGS. 8C, 9C and 10C. FIGS. 8E, 9E and 10E show an overnight feminine pad having the highest absorbency level of the corresponding array. It can be seen that as the performance level of each pad increases, the total surface area of the printed pattern of each pad also correspondingly increases.

Measurement of the total surface area of the pattern coverage can be measured by any means known to one of ordinary skill in the art. One such method, by way of example only, includes the use of a computer scanner with appropriate software. Other means known to those skilled in the art are also suitable. The performance identification device (i.e., the pattern) can assist a user in selecting a pad having a performance value that is desired for a particular menstrual flow. In some aspects, the total surface area of the pattern of a second article in the array may be at least 5% higher than the total surface area of the pattern of a first article having the next lower level of performance, such as at least 10% higher, or at least 30% higher, or at least 50% higher, or more to provide improved benefits. In some aspects, the total surface area of the pattern of a second article in the array may be 10%-800% higher than the total surface area of the pattern of a first article having the next lower level of performance.

In some aspects, the amount of ink utilized for a given printed pattern can be determined via ink volume measurement during the manufacturing process. For example, Table 1 below shows the amount of ink utilized for the print patterns of several exemplary related feminine pads in an array, as each pad in the array increases in absorbency level, as measured by the Absorbent Capacity Test:

TABLE 1

| | | | Specific | |
|---|---|---|---|---|
| | Saturation | Retention | Retention | |
| | Capacity | Capacity | Capacity | Ink |
| Feminine Pad | (g) | (g) | (g/g) | (ml/pad) |
| Invention - Invisible | 9.1 | 6.4 | 6.0 | 0.567 |
| Feel Liner | | | | |
| Invention - Body | 18.2 | 10.2 | 7.0 | 5.495 |
| Contour Liner | | | | |
| Invention - Ultrathin | 63.8 | 39.1 | 7.6 | |
| Invention - Heavy Flow | 85.5 | 55.7 | 7.8 | 13.270 |
| Invention - Overnight | | | | 17.374 |

It can be seen from Table 1 that as performance levels increase, the amount of ink utilized also increases. Further discussion of performance parameters and test methods, as well as other features of the present invention, can be found in U.S. Patent Publication No. 2005/0059942 to Krautkramer et al. and U.S. Patent Publication No. 2008/0234645 to Dodge, each of which is incorporated herein by reference in a manner that is consistent herewith.

In some aspects of the invention, a first disposable personal care absorbent article and a second disposable personal care absorbent article of an array each comprise a printed pattern having the same color, where the color is bold, brilliant and/or vivid. Such colors can promote an environment of fun, and teach away from the current culture of sensitivity, privacy and embarrassment that is associate with current feminine care products. Rather, the patterns of the present invention evoke a positive emotional response such as fun, pride, openness, and the like. For example, with reference to FIGS. 8A-10E, the “fun” print patterns are creative and artistic, particularly when combined with a color that is bold, brilliant and/or vivid. In some aspects, the patterns are continuous in nature (as opposed to iconic in nature) and/or in free-form.

While print patterns of current feminine pads tend to be functional in nature (e.g., to provide a target area, a placement aid, or a perception of depth), the printed patterns of the present invention have an aesthetic element which evokes a positive emotional benefit, such as fun and/or pride for instance. For example, in some aspects, the printed pattern can coordinate with the wearer's outer-garments. In other aspects, the print pattern can be an artistic design, similar to a person viewing a painting at an art gallery, for example. In some aspects, the print patterns are in the form of designs, rather than characters, figures, numerals or words. In further aspects, the print patterns further have a non-functional element to them with respect to the article. Ideally, the patterns of the present invention result in the user feeling comfortable with herself, her body, the fact that she is menstruating, and desirably that she is comfortable talking openly to others about herself and the articles that she is using.

In the case of printed patterns, such patterns can be disposed on a substrate by means well known in the art, including but not limited to printing (e.g., ink-jet, or other forms of printing in which the ink is delivered without a roll or other device impacting or contacting the substrate; gravure, or other forms of printing in which a roll or other device contacts the substrate mechanically). Printing may be accomplished as part of a series of unit-operations employed to make the product, such as an article, wrapper, packaging, etc. (i.e., a printing operation that is “in-line” or part of the sequence of unit operations used to make the item). Alternatively, a substrate may be printed first, then used as a material input to the sequence of operations used to make the product (i.e., a printing operation that is “off-line” from the process used to make the product). Of course other sequences of unit operations, whether in-line or off-line, may be used, so long as the finished product comprises a substrate on which is disposed a pattern of the present invention (e.g., evokes a positive emotional response). Further discussion of printing, as well as other features of the present invention, can be found in U.S. patent application Ser. No. 12/533,458 to Krautkramer et al., which is incorporated herein by reference in a manner that is consistent herewith.

Printed patterns of the present invention can include a single color, multiple colors and/or multiple shades of a single color. The colors can be coordinated or non-coordinated. Furthermore, in some aspects, patterns disposed on the viewing surface of a wrapper 60 and/or a package 70 can be the same as, or visually coordinated with, the pattern of the articles disposed within. In other aspects, the coordination of the pattern of an article with the pattern of a corresponding wrapper for example, allows the manufacturer to use a relatively see-through packaging material without sacrificing the ability to communicate the contents of the package 70. For example, a relatively light basis weight non-woven or film material can be used for the packaging, thereby reducing the overall costs of the product. Further discussion of printing, as well as other features of the present invention, can be found in U.S. Patent Publication No. 2005/0154365 to Zander et al., which is incorporated herein by reference in a manner that is consistent herewith.

In some aspect, patterns can also be described by their location, extent, shape, and orientation. Some overall patterns may have smaller patterns contained within larger or broader patterns. The pattern may be the result of printing, and may further include embossing. Further discussion on the use of embossing can be found in U.S. Pat. No. 4,518,451 to Luceri et al. and U.S. Pat. No. 4,623,340 to Luceri et al., incorporated herein by reference in a manner that is consistent herewith. In some aspects, the pattern can also be determined from the visible spatial, spectral and angular distribution of light coming from an object. Accordingly, in some aspects, embossing may be utilized to help emphasize or highlight a particular print pattern.

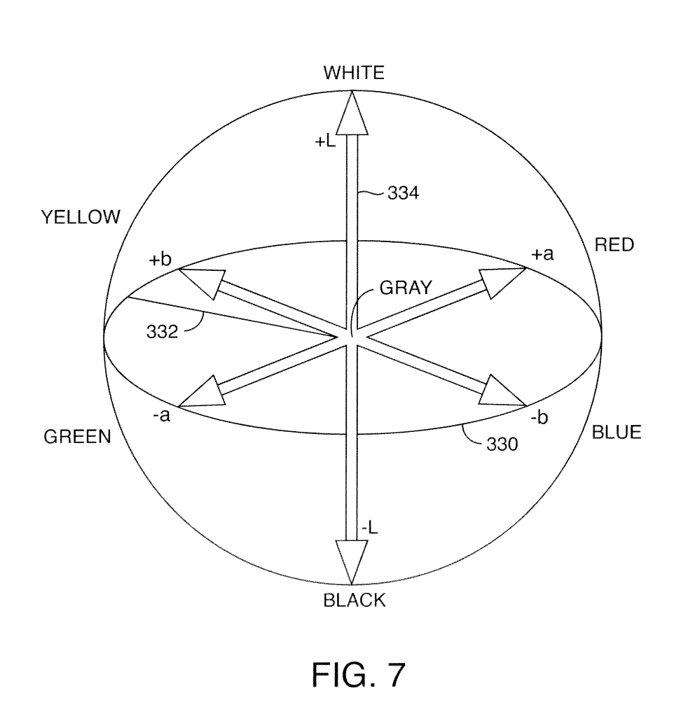

The color of the printed patterns can be described by the level of hue, luminosity (value), and saturation/vividness (chroma). The hue, luminosity and saturation/vividness of a color can be determined by various methods, such as the Color Measurement Test, the Hunter Color Test, or equivalent. Furthermore, a color having a particular hue, luminosity and saturation/vividness, such as measured by the Hunter Color Test, can be applied to an internationally recognized 3-dimensional color chart as would be well-known to those skilled in the art, such as the color chart 310 of FIG. 7 showing the Hunter L,a,b color space, and can be utilized to determine whether a color is the “same” or “different.” For purposes of this invention, a color that is not different is considered to be the same. For example, the hue of a particular color can be applied to the color chart 310 of FIG. 7 along circle 330 having 360 degrees. Thus, two colors are considered to be different herein if they have first and second respective hues that are greater than ±10 degrees apart when viewed along circle 330. In addition, the value (luminosity) of a color can be applied to the color chart 310 along the z-axis 334 between white and black. Luminosity is measured on a scale from white to black. A luminosity of 100% of the maximum of the axis 334 (i.e., the top of the axis) equates to white while a luminosity of 0% (i.e., the bottom of the axis) equates to black. Thus, two colors are considered to be different herein if they have a luminosity difference of greater than 10% of maximum of the z-axis 334. Further, the saturation/vividness (chroma) of a color can be applied to the color chart 310 along the length of the radius (r) 332. Thus, two colors are also considered to be different herein if they have a saturation/vividness difference of greater than 20% of maximum of the radius 332.

In some aspects, desirable colors include those that are bold, vivid and/or brilliant. These colors would typically be viewed as illicit or even taboo under the current culture of discreetness and privacy with respect to feminine care during menstruation. However, suitable colors for the invention include, but are not limited to, non-traditional colors such as brilliant blues, bold purples, vivid greens, bright oranges, reds, and the like. As measured by the Hunter Color Test, colors are considered to be “bold” when they have an L-value of greater than 56 or less than 26, an a*-value of greater than 7 or less than −45, and a b*-value of greater than 46 or less than −8. As measured by the Hunter Color Test, colors are considered to be “vivid” when they have an L-value of greater than 56 or less than 49, an a*-value of greater than 38 or less than −13, and a b*-value of greater than 32 or less than −33. As measured by the Hunter Color Test, colors are considered to be “brilliant” when they have an L-value of greater than 65, an a*-value of greater than 49 or less than −2, and a b*-value of greater than 36 and less than −9.

In some aspects, colors can be identified by a PANTONE identification provided by Pantone LLC, a wholly owned subsidiary of X-Rite, Incorporated, which is well-known to those skilled in the art, and can be found at http://www.pantone.com/pages/pantone/colorfinder.aspx. Accordingly, examples of colors that are “bold” include, but are not limited to, PANTONE: 122U, 219U, 313U, 361U, 1505U and 2735U. Examples of colors that are “vivid” include, but are not limited to, PANTONE: 285U, 306U, 313U, 368U, 373U and 2592U. Examples of colors that are “brilliant” include, but are not limited to, PANTONE: 108U, 232U, 389U and 374U. It is understood that many more colors are also suitable, provided that they meet the L,a*,b* criteria for being bold, vivid and/or brilliant.

As referenced above, in some aspects, the printed patterns of two or more related articles in the array can be visually coordinated. As used herein, the term “visually coordinated,” when referring to the print patterns disposed on the personal care products of the present invention are used to describe how the overall print patterns of an array of two or more related personal care products, when each product is viewed as a whole, are designed to visually match each other, or otherwise belong together with respect to pattern element color and pattern style. Accordingly, two or more personal care products in the array are considered to be visually coordinated when a particular print pattern element in the form of a shape or design is within the criteria for “pattern element color” and for “pattern element style.” As used herein, the color of a particular pattern element of a first disposable personal care absorbent article in an array and the color of the corresponding pattern element of a second disposable personal care absorbent article in the array meet the criteria for pattern element color if they are the “same” (i.e., not “different} as discussed above with respect to hue, luminosity and saturation.

As used herein, a particular pattern of a first disposable personal care absorbent article in an array and the corresponding pattern of a second disposable personal care absorbent article in the array meet the criteria for “pattern element style” if the pattern of each product in the array share pattern elements in the form of shapes or designs such that the user would recognize that the print patterns are thematically related when viewed as a whole. Accordingly, at least a portion of the pattern elements in the overall print patterns of the array should be substantially the same for each product, although such pattern elements can be different sizes and/or mirror images, provided they have substantially the same shape profile.

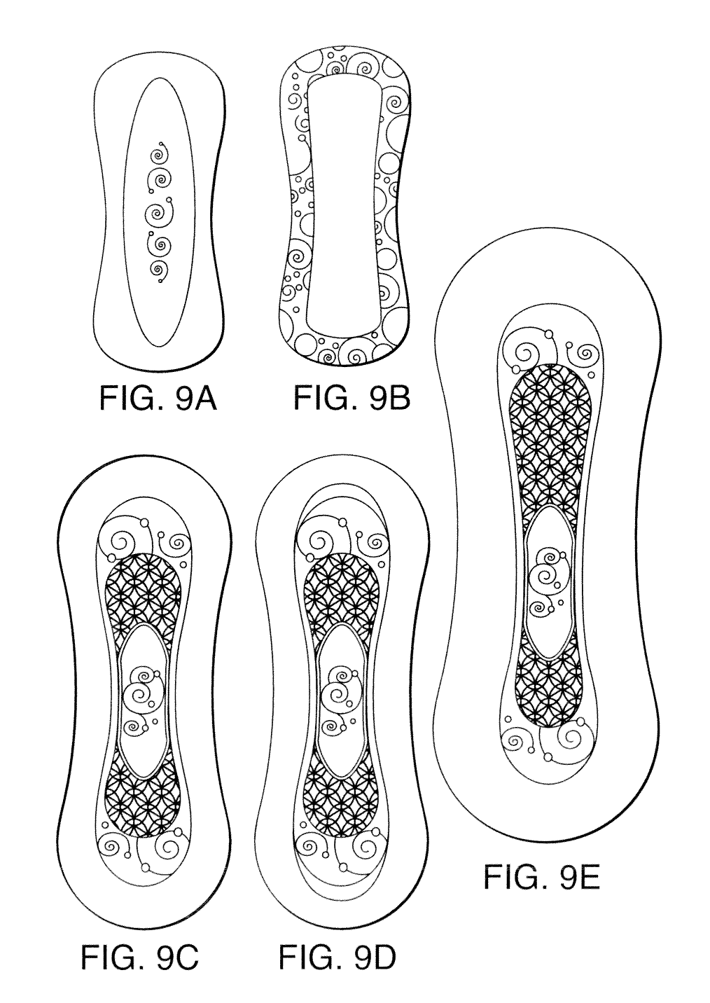

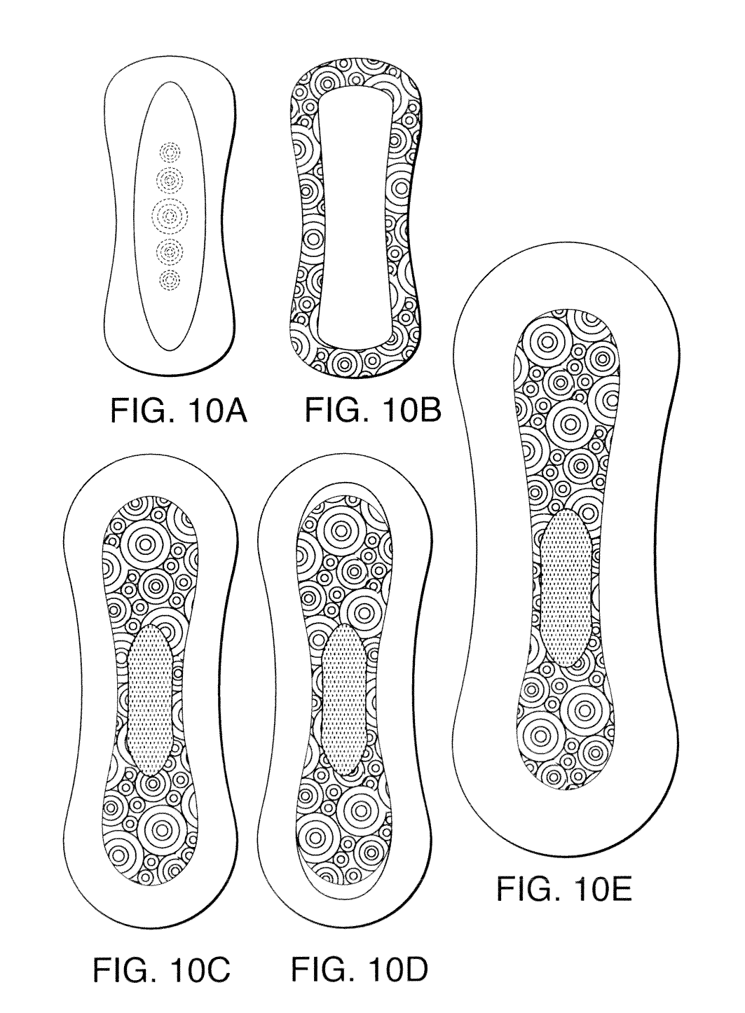

Without wishing to be limited to the specific embodiments listed, and assuming the criteria for pattern element color has been met, the following examples of pattern element styles illustrate print patterns that are visually coordinated:
A first product in the array where the overall print pattern comprises a series of different size longitudinally-extending circle-shaped pattern elements (the coordinating pattern element style of the array), each consisting of a relatively smaller filled circle (i.e., a solid circle) disposed within a relatively larger unfilled circle (i.e., a circle outline); a second product having two series of different size longitudinally-extending circle-shaped pattern elements consisting of a relatively smaller filled circle disposed within a relatively larger unfilled circle and having the same shape profile as the first product; and a third product having three series of different size longitudinally-extending circle-shaped pattern elements consisting of a relatively smaller filled circle disposed within a relatively larger unfilled circle and having the same shape profile as the first product, and further including a semi-circle-shaped pattern element disposed at the longitudinal ends of the product (see e.g., FIGS. 8A-8C).
A first product in the array where the overall print pattern comprises swirls (the coordinating pattern element style of the array) where the overall pattern is located in a center portion of the product; a second product having different size swirls, some of which are mirror images of the first product but having the same shape profile as the first product, and further including unfilled circles, where the overall pattern is disposed in an outer perimeter portion of the product; and a third product comprising different size swirls having the same shape profile as the first product, where the overall pattern is located in the longitudinal end portions of the product, and further comprising an overlapping circle pattern disposed therebetween (see e.g., FIGS. 9A-9C).
A first product in the array where the overall print pattern comprises a series of various size concentric circle-shaped pattern elements (the coordinating pattern element style of the array) where the overall pattern is located longitudinally in a center portion of the product; a second product having the same concentric circle-shaped pattern elements of the first product where the overall pattern is located in an outer perimeter portion of the product; and a third product having the same concentric circle-shaped pattern elements of the first product, where the overall pattern has a dog-bone profile and is located in the transverse center portion of the product and extends towards the longitudinal end portions of the product, and further comprising a mesh-like pattern disposed in the central portion of the product (see e.g., FIGS. 10A-10C).
At least two products in an array, both having overall print patterns comprising various size swirl-shaped pattern elements having the same shape profile (a first coordinating pattern style of the array), and various size butterfly-shaped pattern elements (a second coordinating pattern style of the array) having the same shape profile.
At least two products in an array, both having overall print patterns comprising various size outlined circles-inside-of-circles-shaped pattern elements having the same shape profile (a first coordinating pattern style of the array), and various size outlined flowers-inside-of-flowers-shaped pattern elements (a seocnd coordinating pattern style of the array).
At least two products in an array, both having overall print patterns comprising various size concentric square-shaped pattern elements (a first coordinating pattern style of the array) and various size RUBIK'S CUBE-shaped pattern elements (a second coordinating pattern style of the array).
   Other visually coordinated patterns are also suitable for the invention, and would be readily apparent to those skilled in the art.

To further illustrate, again assuming the criteria for pattern element color has been met, the following examples illustrate print patterns that are not visually coordinated:
A first product in the array with an overall print pattern comprising circle-shaped pattern elements and a second product with an overall print pattern comprising oval-shaped pattern elements, but without also having circle-shaped pattern elements with the same shape profile as the first product,
A first product in the array with an overall print pattern comprising long, thin heart-shaped pattern elements and a second product with an overall print pattern comprising short, wide heart-shaped pattern elements, but without also having long, thin heart-shaped pattern elements with the same shape profile as the first product,
A first product in the array with an overall print pattern comprising paisley-shaped pattern elements that are filled and a second product with an overall print pattern comprising paisley-shaped pattern elements with the same shape profile as the first product, but that are unfilled.

The patterns of the invention can be disposed onto various locations within an absorbent article, provided that they are visible to the user as defined above. For example, the article may comprise one or more components, including but not limited to, an absorbent core, a topsheet, a backsheet, a surge layer, a distribution layer, a shaping layer, and the like. Each component has a body-facing surface and a garment-facing surface. The pattern of the invention can be disposed on at least one of either surface on any one or more of the components of the article, provided it is visible to the user. For example, in one aspect, the pattern is disposed on the body-facing surface of a topsheet. In another aspect, the pattern is disposed on the garment-facing side of a topsheet. In still another aspect, the pattern is disposed on a body-facing side of the absorbent core. In yet another aspect, the pattern is disposed on the body-facing side of a surge layer. In still another aspect, the pattern is disposed on the garment-facing side of a backsheet. One of ordinary skill in the art will readily recognize that various other locations and combinations thereof are also well within the scope of the invention.

In some desirable aspects, the pattern can be continuous. In accordance with the definition provided above, a continuous pattern is a pattern that a user perceives as being uninterrupted. For example, a continuous pattern can appear to the user as a barrier to fluid movement, and can provide the user with an additional level of perceived security. For instance, if the performance cue is “protection” for example, the continuous pattern should not only communicate the performance level, but should also reinforce that the product will protect the user during menstruation. In one example, the continuous pattern can be disposed along the perimeter of the article surface.

In some aspects, the pattern can further include pattern elements that appear as moving objects on the outside of the article. In other aspects, the pattern can further include pattern elements that disappear or appear upon fluid insult of the article, enticing the user to perhaps have fun looking at the article, even after use. In still other aspects, the print pattern can further include an optical illusion. Such optical illusions can provide an indication of performance characteristic, evoke a positive emotional response, combinations thereof, or neither. In some aspects, the optical illusion can draw the gaze of a user to a certain area of the article. By drawing the gaze of the user, the optical illusion may highlight a benefit associated with the article and/or guide the user in proper application of the article. The optical illusion chosen also enables intuitive highlighting of benefits provided depending on applications for the article. Exemplary benefits include, but are not limited to, fun, comfort, convenience, absorbency, softness, thickness, and the like. For example, the optical illusion can identify a location on the article where a medication is disposed, or a location on the article where a user is directed to apply a stimulus (e.g., urine, menses, pressure) to activate or utilize a substance disposed on the article.

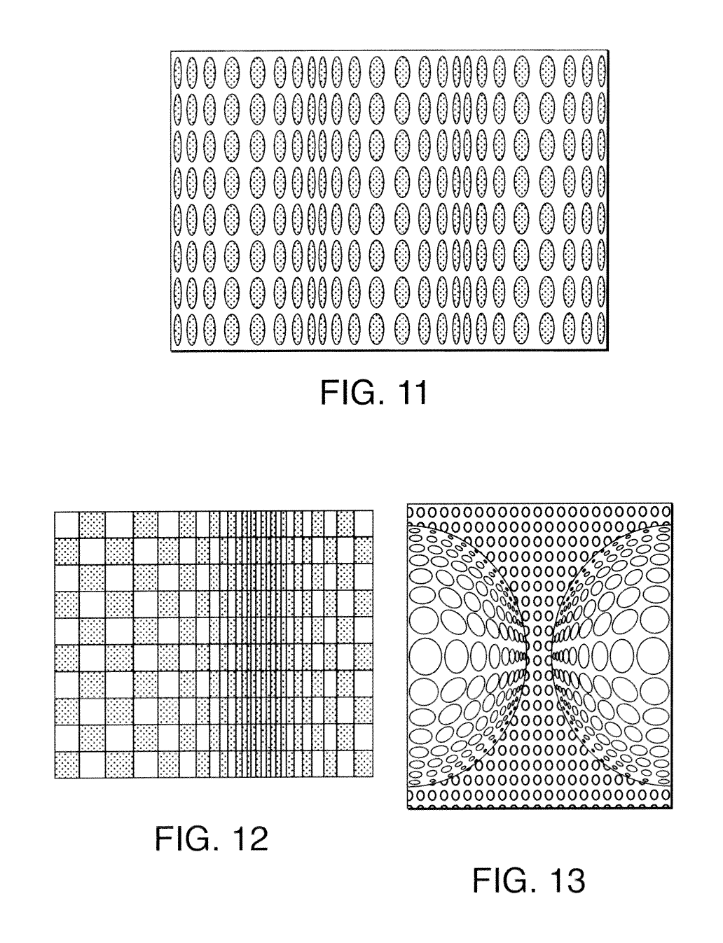

Various optical illusions are contemplated and are suitable for creating the illusions on the article regardless of any particular theory or technique employed with respect to arranging any pattern elements in a pattern to create the illusion. In some aspects, the pattern formed by a plurality of pattern elements can create the optical illusion. With reference to FIGS. 11-13, by way of example, lines with varying widths and/or varying spacing between adjacent lines provide a selected arrangement (i.e., part of the overall pattern) which creates depth/height illusions. Similarly, changing the size and spacing of repeated closed figures, such as an oval, produces an arrangement that also creates elevation based illusions. Particular types of suitable illusions, alone or in combination, include geometric, spiral, anomalous motion, rotational, color change, peripheral drift, positive after image blurs, scintillation grids, stereopsis and chromostreopsis, contraction and expansion, contrast polarities, convection, and chromatic assimilation. Additionally, optical illusions of motion may further implement optical illusionary elevation deviation relative to an exterior surface of the article either in separate illusions or one integral illusion. This may be desirable in the case of an array of ultrathin pads having different absorbency levels which structurally looks similar. For instance, a pad with a relatively higher absorbency than a pad with a relatively lower absorbency can include an optical illusion that results in the user perceiving that the pad having a higher absorbency also has a higher thickness than the pad having a lower absorbency, when in reality the pads each have a thickness that is substantially equal.

With further reference to FIGS. 11-13, the illusion provided by the graphical elements 1301, 1401 and 1501 can create a perception of movement to a location of the article along with thickness and depth that simulates fluid flowing and moving through or deeper into the article. These perceptions correlate to enhancements in protection and comfort to the user, for example. Articles offering greater performance, such as absorbency or softness for example, may utilize an illusion that appears to have more movement and/or greater elevation deviation relative to an illusion on articles that provide less performance. Patterns that provide graphical illusions may also, or alternatively, be disposed on wrappers 60 containing the articles 20, or on the package 70 itself. In some aspects, a pattern on the package 70 may be related to the pattern formed on the article(s) 20, thereby reinforcing the illusion visible on the article 20. Alternatively, the wrapper 60 or package 70 may include a different illusionary pattern of pattern elements to provide a further element of fun to the user, and/or can assist with selection of the article by the user.

In some aspects, some pattern elements are initially at least partially invisible or lighter and become visible or darker when exposed to some stimulus or substance (e.g., urine and/or complex fluids). Further, some pattern elements may be initially visible as a first color and become invisible, lighter or a second color when exposed to the stimulus or substance. For example, suitable compositions can include an ink that creates the pattern element and changes color when exposed to stimulus or substance. Further discussion of illusions, as well as other features of the present invention, can be found in U.S. patent application Ser. No. 11/957,163 to Sullivan et al., which is incorporated herein by reference in a manner that is consistent herewith.

In some aspects, a communication means pertaining to the array of articles can be provided to communicate information to the user. In some desirable aspects, the communication means is a visual aid. Although discussed primarily in terms of a visual aid, it is to be understood that the communication means may be in other suitable forms.

In some aspects, a word or image depicting the consumer's current product choice may be highlighted or otherwise emphasized in the communication means. In instances where the communication means provides a recommendation of a future product choice, the depiction of the recommended future product choice may likewise be highlighted or emphasized.

In some aspects, the communication means can comprise at least one scale of reference. The scale of reference can include a depiction of a common characteristic of the array of related absorbent products, and can provide a means by which the comparison of groups of related absorbent products may be made. The common characteristic depicted in the scale of reference may be related to a consumer need or desire, and may include, for example, a benefit, an attribute, a feature, or combinations thereof. Typically, the scale of reference depicts a range of values of a certain common characteristic shared by one or more groups of related products depicted in the communication means. For example, in one aspect, the scale of reference may depict a range of values for absorbent capacity. Typically, the values are ordered marks arranged on the scale at a fixed interval. The values may be depicted using any suitable means including, for example, a letter, a number, a word, a symbol, a picture, or combinations thereof.

The communication means can include one scale of reference, or alternately, can include two or more scales of reference. Using two or more scales of reference can be advantageous if multiple comparisons are to be made between the related groups of absorbent products. Although each scale typically depicts a different product characteristic, it is also contemplated that a visual aid may comprise two or more scales, with each scale depicting the same product characteristic.

In some aspects, products depicted in each region of the visual aid are positioned within the region so that they line up in some manner with corresponding markings on the scale of reference. For example, referring to FIG. 18, an exemplary communication means, which could be present on a package 70 for example, is depicted. A first region 2131 of the communication means comprises a depiction of a group of products related according to one product category, e.g., print pattern. A second region 2132 comprises a scale of reference that depicts a common characteristic of the absorbent products depicted in the first region 2131. The scale of reference depicts a range of values for a particular product characteristic, such as absorbent capacity, that is represented on the scale as ordered marks at a fixed interval 2126, where “X” represents an alpha-numeric value. As can be seen from FIG. 18, the absorbent products depicted in the first region 2131 and in the second region 2132 are positioned within their respective regions so that the individual products line up with the value on the scale that represents the product's particular performance value. In this manner, a consumer may easily compare how the absorbency of a product in one product category compares to the absorbency of a product in another product category; e.g., how the absorbent capacity of a particular feminine care product compares to the absorbent capacity of another particular feminine care product. For example, a consumer may locate her current product choice in the second region 2132 of the communication means and, referring to the visual depiction of the print pattern of reference in the first region 2131, may locate a product choice that has a similar absorbent capacity as her current product choice.

A similarly arranged communication means may be used to compare other absorbent product characteristics. For example, in another embodiment, a third region 2133 may comprise a scale of reference that depicts a different common characteristic (e.g., a different benefit, attribute, and/or feature) of the absorbent products depicted in the first region 2131. This different scale of reference depicts a range of values for a particular product characteristic that is represented on the scale as ordered marks at a fixed interval 2126, where “Y” represents an alpha-numeric value.

In some aspects, the communication means may comprise additional regions, each depicting a different group of related products and one or more regions comprising one or more scales of reference. Accordingly, although the communication means illustrated in FIG. 18 depicts only three different regions, it is contemplated that the communication means may further comprise four, five, or more additional regions that each depict a different group of related products. In instances where the communication means comprises numerous different regions each depicting a different group of related products, it may be advantageous for the communication means to comprise two or more regions each comprising the same scale of reference. The regions comprising the scale of reference are preferably positioned adjacent to different regions depicting the groups of related products, in order to allow for easier comparison of the products using the scale of reference.

The communication means described herein may be present on a variety of different media. For example, the communication means may be present on any form of printed media including, for example, newspapers, pamphlets, magazines, displays, labels and various forms of advertisements, such as billboards, posters, banners, flyers, instructions, other forms of direct-mail or out-of-home media, and the like. The communication means may also be present on an electronic medium, such as a television, a computer screen, personal data assistant, cellular telephone, an internet display, MP3 players, hand-held video units, and the like.

In one particular aspect, the communication means may be present as part of an in-store display or shelf set located in the general proximity of an absorbent product depicted in the communication means. In one example of this embodiment, the display may be located between two or more shelves of products, with one or more shelves having one group of related products depicted in the communication means, and another shelf or shelves having another group of related products depicted in the communication means. The scale of reference provided by the communication means allows for easy comparison between the groups of related products. The in-store display may be some form of printed material, or optionally, may be an interactive display, as described below.

In some aspects, an absorbent article 20, a wrapper 60, and/or a package 70 may comprise the communication means. For example, the communication means may be packaged with a plurality of absorbent articles 20 and contained in the hollow interior of the package 70 and/or may be wrapped with an individual absorbent article 20 and contained in the hollow interior of the wrapper 60. Alternately or in addition, the communication means may be on a removable release material, such as a removable label, peel strip, tear strip, or other removable layer that may be attached to the absorbent article 20, the wrapper 60, and/or the package 70. For example, the communication means may be located on an exposed surface of a removable release material, which is arranged to face outwardly toward the consumer. Alternately, the communication means may be positioned on an interior surface of the removable release material, such that it is exposed to the consumer only upon removal of the removable release material. In some aspects, the communication means may be positioned on a removable release material 40 used to cover a garment adhesive 38 used to adhere an absorbent article 20 to the undergarments of a wearer.

In some aspects, the communication means is present on the exterior of a package 70. In this instance, the communication means may be created by marking the package 70 so that the communication means is visually perceived and understandable to the consumer. The markings can be applied on the package 70 by printing, embossing, die-cutting, application of a discrete label on which the communication means is located or, in general, by any other physical change in the form or character of the package 70 such as to make the markings visually noticeable and understandable to the consumer. Optionally, there may be cut lines and/or perforations around the communication means located on the package 70, to enable a consumer to remove the communication means from the package 70 or wrapper 60.

In instances where the communication means is found on an absorbent article 20, a wrapper 60, and/or on a package 70, the absorbent article 20 will typically be one of the products depicted on the communication means. The scale or scales of reference provided by the communication means allows a consumer to compare a characteristic(s) of that particular absorbent article 20 to other absorbent articles depicted on the communication means. In some aspects, the depiction of the absorbent article may be highlighted or otherwise emphasized for easier identification of the absorbent article 20 on the communication means. Optionally, the communication means may be removed from the absorbent article 20, wrapper 60, and/or package 70 and taken with the consumer to the store to aid the consumer in the selection of a new absorbent product or in the transition to a new absorbent product.

The present disclosure also provides various methods for assisting a consumer in transitioning from a current absorbent product to a future absorbent product. Such methods are typically interactive (i.e., the consumer has input on the selection and/or formation of the communication means that is provided to the consumer). In this manner, the consumer is provided with a communication means specifically designed for his or her needs.

Generally, such methods involve receiving personal information from the consumer about at least one need of the consumer and providing the consumer with a communication means, such as those described herein, that is customized for the consumer. More particularly, the method typically comprises receiving personal information from the consumer about at least one need of the consumer, receiving information from the consumer about an absorbent product currently used by the consumer, selecting a first group of related absorbent products that comprise the current absorbent product choice of the consumer, optionally selecting at least one additional group of related absorbent products, and providing the consumer with a communication means.

The information is typically received from the consumer through an interactive means. For example, the consumer may input information into a user interface (e.g., speaking the information, selecting the appropriate information from a menu of items in a graphical user interface, typing in a code, product name, or other information, pressing a button specific to a certain piece of information, using an interactive menu-driven phone system, and the like). A user interface may include, for example, a mobile, cellular telephone; an electronic, personal data assistant; an electronic computer configured with an operative user-interface, such as a graphical user interface; a website; an electronic display; and the like. In some aspects, an interactive computer can prompt a consumer to input several key pieces of information about his or her needs and current product choice. Further discussion of the communication means, as well as other features of the present invention, can be found in U.S. patent application Ser. No. 11/304,342 to Woltman et al., which is incorporated herein by reference in a manner that is consistent herewith.

The present invention may be better understood with reference to the following examples.

### EXAMPLES

An array of feminine pads were provided to a group of test subjects. The test subjects were females age 14 to 49 who use at least six or more ultra thin winged pads per month. This study was a positioned comparison use test using a 10-cell Balanced Incomplete Block (BIB) design. Cells were balanced by age, brand used most often, and flow (light, regular, heavy). All products tested were regular absorbency ultra thins with wings. The invention samples were similar to those depicted in FIGS. 8A-10E, but with the addition of wings. Some of the pads were printed with a vivid blue having a PANTONE Identification of 284 U (hereinafter referred to as Vivid Blue), while others were printed with a bold purple color having a PANTONE Identification of 2715 U (hereinafter referred to as Bold Purple). The test subjects were also provided with product that is currently commercially available as part of the comparison test (ALWAYS Ultra Thin with Flexi-Wings). All of the articles were then viewed and tested by the test subjects and the results can be seen in Tables 2 and 3 below:

TABLE 2

| | | Invention: |
|---|---|---|
| | Current Pads | Vivid Blue |
| Visual Assessment | (%) | (%) |
| Looks like it will protect me better | 48 | 52 |
| from leaks | | |
| Looks more comfortable | 32 | 68 |
| Looks higher in quality | 37 | 63 |
| Has better overall appearance | 25 | 75 |
| Looks more appropriate for someone my age | 46 | 54 |
| Looks more like a product that fits | 40 | 60 |
| my lifestyle | | |
| Looks like it will do a better job of | 44 | 56 |
| keeping me feeling clean | | |
| Looks like it will be more discreet to wear | 56 | 44 |
| Looks more new and different compared to | 20 | 80 |
| other pads | | |

TABLE 3

| | | Invention: |
|---|---|---|
| | Current Pads | Bold Purple |
| Visual Assessment | (%) | (%) |
| Looks like it will protect me better | 34 | 66 |
| from leaks | | |
| Looks more comfortable | 30 | 70 |
| Looks higher in quality | 25 | 75 |
| Has better overall appearance | 16 | 84 |
| Looks more appropriate for someone my age | 36 | 64 |
| Looks more like a product that fits | 32 | 68 |
| my lifestyle | | |
| Looks like it will do a better job of | 30 | 70 |
| keeping me feeling clean | | |
| Looks like it will be more discreet to wear | 56 | 44 |
| Looks more new and different compared to | 10 | 90 |
| other pads | | |

It can be seen from Tables 2 and 3 that pads having patterns of the present invention had a significantly higher preference to current pads in the market. It is also notable that the rating for “Looks like it will be more discreet to wear” was lower for pads having patterns of the present invention than the ratings for current pads in the market. This demonstrates that the “fun” patterns of the present invention are considered to be less discreet than products of the current culture, and further demonstrates a break from the current cultural paradigm.

The invention also includes a method for visually identifying the performance level of disposable personal care absorbent articles. The method includes a) providing a first disposable personal care absorbent article having a body-facing surface and a garment-facing surface; b) providing a second disposable personal care absorbent article having a body-facing surface and a garment-facing surface; and c) disposing a printed pattern that is visible to a user on at least one of the body-facing surface or the garment-facing surface on each of the first disposable personal care absorbent article and the second disposable personal care absorbent article; where the first disposable personal care absorbent article and second disposable personal care absorbent article are related; where the second disposable personal care absorbent article has a performance value that is greater than the first disposable personal care absorbent article; and where the total surface area of printed pattern of the second disposable personal care absorbent article is greater than the total surface area of the printed pattern of the first disposable personal care absorbent article.

It will be appreciated that details of the foregoing examples, given for purposes of illustration, are not to be construed as limiting the scope of this invention. Although only a few exemplary embodiments of this invention have been described in detail above, those skilled in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the desirable embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

What is claimed is:

1. A system for visually identifying the performance level of feminine care pads comprising:
a first feminine care pad and a second feminine care pad, each having a body-facing surface, a garment-facing surface, and a printed pattern that is visible to a user on at least one of the body-facing surface or the garment-facing surface;
wherein the first feminine care pad and the second feminine care pad are related;
wherein the second feminine care pad has a higher absorbent capacity level than the first feminine care pad, as measured by the Absorbent Capacity Test; and
wherein the total printed surface area of the printed pattern of the second feminine care pad is greater than the total printed surface area of the printed pattern of the first feminine care pad.

2. The system of claim 1 wherein the total surface area of the printed pattern of the second feminine care pad is at least 5% greater than the total surface area of the printed pattern of the first feminine care pad.

3. The system of claim 2 wherein the total surface area the printed pattern of the second feminine care pad is 30%-800% greater than the total surface area of the printed pattern of the first feminine care pad.

4. The system of claim 1 wherein the printed pattern of the first feminine care pad and the printed pattern of the second feminine care pad are the same color.

5. The system of claim 1 wherein the printed pattern of the first feminine care pad and the printed pattern of the second feminine care pad each comprise a color that is bold, vivid or brilliant.

6. The system of claim 1 wherein the printed pattern of the first feminine care pad and the printed pattern of the second feminine care pad are visually coordinated.

7. The system of claim 1 wherein each of the first feminine care pad and the second feminine care pad further comprise embossing, wherein the total surface area of the printed pattern and embossing of the second feminine care pad is greater than the total surface area of the printed pattern and embossing of the first feminine care pad.

8. The system of claim 1 wherein the printed pattern of the first feminine care pad and the printed pattern of the second feminine care pad further comprise an optical illusion.

9. The system of claim 1 wherein the printed pattern of the first feminine care pad and the printed pattern of the second feminine care pad each comprise multiple shades of the same color.

10. The system of claim 1 wherein the first feminine care pad and the second feminine care pad each comprise a wrapper, wherein each respective wrapper includes a printed pattern that is substantially the same as the printed pattern of the corresponding feminine care pad.

11. The system of claim 1 further comprising a first package and a second package, wherein the first package contains a plurality of the first feminine care pad and includes a printed pattern that is visually coordinated with the printed pattern of the first feminine care pad, and wherein the second package contains a plurality of the second feminine care pad and includes a printed pattern that is visually coordinated with the printed pattern of the second feminine care pad.

12. The system of claim 1 further comprising a communication means including at least one scale of reference.

13. The system of claim 1 wherein the absorbent capacity is saturated capacity.

14. The system of claim 1 wherein the absorbent capacity is retention capacity.

15. A system for visually identifying the performance level of feminine care pads comprising:
a first feminine care pad and a second feminine care pad, each having a body-facing surface, a garment-facing surface and a pattern that is visible to a user on the body-facing surface;
wherein the first feminine care pad and the second feminine care pad are related;
wherein each pattern comprises printing;
wherein the printing of the first feminine care pad and the printing of second feminine care pad are the same color, wherein the color is bold, brilliant or vivid;
wherein the saturation capacity of the second feminine care pad is greater than the saturation capacity of the first feminine care pad, as measured by the Absorbent Capacity Test; and
wherein the total surface area of the pattern of the second feminine care pad is at least 10% greater than the total surface area of the pattern of the first feminine care pad.

16. The system of claim 15 wherein the total surface area of the pattern of the second feminine care pad is at least 50% greater than the total surface area of the printing of the pattern of the first feminine care pad.

17. The system of claim 15 wherein the first feminine care pad and the second feminine care pad each further comprise a wrapper, wherein each respective wrapper includes a printed pattern that is substantially the same as the pattern of the corresponding feminine care pad.

18. The system of claim 15 further comprising a first package and a second package, wherein the first package contains a plurality of the first feminine care pad and includes a printed pattern that is visually coordinated with the pattern of the first feminine care pad, and wherein the second package contains a plurality of the second feminine care pad and includes a printed pattern that is visually coordinated with the pattern of the second feminine care pad.

19. The system of claim 15 further comprising a communication means including at least one scale of reference.

20. A method for visually identifying the performance level of disposable personal care absorbent articles comprising:
a) providing a first disposable personal care absorbent article having a body-facing surface and a garment-facing surface;
b) providing a second disposable personal care absorbent article having a body-facing surface and a garment-facing surface; and
c) disposing a printed pattern that is visible to a user on at least one of the body-facing surface or the garment-facing surface on each of the first disposable personal care absorbent article and the second disposable personal care absorbent article;
wherein the first disposable personal care absorbent article and second disposable personal care absorbent article are related;
wherein the second disposable personal care absorbent article has a performance value that is greater than the first disposable personal care absorbent article; and
wherein the total surface area of the printed pattern of the second disposable personal care absorbent article is greater than the total surface area of the printed pattern of the first disposable personal care absorbent article.

21. The method of claim 20 wherein the printed pattern of the first disposable personal care absorbent article and the printed pattern of the second disposable personal care absorbent article are visually coordinated.

22. The method of claim 20 wherein the printed pattern of the first disposable personal care absorbent article and the printed pattern of the second disposable personal care absorbent article each comprise a color that is bold, vivid or brilliant.

23. The method of claim 20 wherein the performance value is saturation capacity, as measured by the Absorbent Capacity Test.

24. The method of claim 20 wherein the personal care absorbent articles are feminine care pads.
